(19) United States
(12) Patent Application Publication
(10) Pub. No.: US 2026/0011415 A1
(43) Pub. Date: Jan. 8, 2026

(54) SYSTEM AND METHOD FOR AUTOMATED FILE REPORTING

(71) Applicant: WISEDOCS INC., Toronto (CA)

(72) Inventors: Connor ATCHISON, Elora (CA); Rajiv ABRAHAM, Toronto (CA); Wei SUN, Kitchener (CA); Ryan JUGDEO, Toronto (CA); Leo ZOVIC, North York (CA)

(21) Appl. No.: 18/880,026

(22) PCT Filed: Jul. 28, 2023
(86) PCT No.: PCT/CA2023/051024

§ 371 Date: Dec. 30, 2024

Related U.S. Application Data
(60) Provisional application No. 63/393,044, filed on Jul. 28, 2022.

(51) Int. Cl. G06F 16/31; G06F 16/35; G06V 10/40; G06V 10/82; G06V 30/10; G16H 10/60; G16H 15/00

(52) U.S. Cl. CPC G16H 10/60; G06F 16/35; G06V 10/40; G06V 10/82; G16H 15/00

(57) ABSTRACT

A document index generating system and method are provided. The system comprises a processor and a memory storing a sequence of instructions which when executed by the processor configure the processor to perform the method. The method comprises preprocessing a plurality of pages into a collection of data structures, classifying each preprocessed page into at least one document type, segmenting groups of classified pages into documents, and generating a page and document index for the plurality of pages based on the classified pages and documents. Each data structure comprises a representation of data for a page of the plurality of pages. The representation comprises at least one region on the page, comprising for each page, normalizing the plurality of pages into a collection of images and a collection of plain text, obtaining vision features from the collection of images and processing the collection of plain text.

### FIELD

The present disclosure generally relates to the field of automated reporting, and in particular to a system and method for automated file reporting.

### INTRODUCTION

When performing a task that requires the organization of a large file (for example, when assessing an insurance claim, an assessment officer must review the health record of a patient or claimant), the large file may comprise several thousand pages, causing delays or missed information. Sometimes, the files (e.g., health records) may be compiled manually into a report, sometimes with comments from the assessor who prepared the report.

### SUMMARY

In accordance with an aspect, there is provided a document index generating system. The system comprises at least one processor and a memory storing a sequence of instructions which when executed by the at least one processor configure the at least one processor to preprocess a plurality of pages into a collection of data structures, classify each preprocessed page into at least one document type, segment groups of classified pages into documents, and generate a page and document index for the plurality of pages based on the classified pages and documents. Each data structure comprises a representation of data for a page of the plurality of pages. The representation comprises at least one region on the page, comprising for each page, normalizing the plurality of pages into a collection of images and a collection of plain text, obtaining vision features from the collection of images and processing the collection of plain text.

In accordance with another aspect, there is provided a computer-implemented method for generating a document index. The method comprises preprocessing a plurality of pages into a collection of data structures, classifying each preprocessed page into at least one document type, segmenting groups of classified pages into documents, and generating a page and document index for the plurality of pages based on the classified pages and documents. Each data structure comprises a representation of data for a page of the plurality of pages. The representation comprises at least one region on the page, comprising for each page, de-noising the plurality of pages into a collection of images and a collection of plain text, obtaining vision features from the collection of images and processing the collection of plain text.

In various further aspects, the disclosure provides corresponding systems and devices, and logic structures such as machine-executable coded instruction sets for implementing such systems, devices, and methods.

In this respect, before explaining at least one embodiment in detail, it is to be understood that the embodiments are not limited in application to the details of construction and to the arrangements of the components set forth in the following description or illustrated in the drawings. Also, it is to be understood that the phraseology and terminology employed herein are for the purpose of description and should not be regarded as limiting.

Many further features and combinations thereof concerning embodiments described herein will appear to those skilled in the art following a reading of the instant disclosure.

### DESCRIPTION OF THE FIGURES

Embodiments will be described, by way of example only, with reference to the attached figures, wherein in the figures:

FIG. 1 illustrates, in a schematic diagram, an example of an automated medical report system platform, in accordance with some embodiments;

FIG. 2 illustrates, in a flowchart, an example of a method of generating an index of a document, in accordance with some embodiments;

FIG. 3 illustrates, in a flowchart, another example of generating an index of a document, in accordance with some embodiments;

FIG. 4 illustrates, in a process flow diagram, an example of a method of preprocessing a PDF document, in accordance with some embodiments;

FIG. 5 illustrates, in a screenshot, an example of a portion of a PDF page in a PDF document, in accordance with some embodiments;

FIG. 6A illustrates, in a flowchart, another example of a method for classifying pages, in accordance with some embodiments;

FIG. 6B illustrates, in a flowchart, an example of a method for determining a document type from pages with unknown document formats, in accordance with some embodiments;

FIG. 7 illustrates, in a flowchart, an example of a method of generating an index (or a table of contents) from the output of the classification component, in accordance with some embodiments;

FIG. 8A illustrates, in a flowchart, an example of summarizing a document, in accordance with some embodiments;

FIG. 8B illustrates, in a flowchart, a method of chunk splitting, in accordance with some embodiments;

FIG. 9 illustrates, in a flowchart, another method of summarizing a document, in accordance with some embodiments;

FIG. 10 illustrates, in a schematic, an example of a system environment, in accordance with some embodiments;

FIG. 11 illustrates, in a screen shot, an example of an index, in accordance with some embodiments;

FIG. 12 illustrates another example of an index, in accordance with some embodiments;

FIG. 13 illustrates, in a screen shot, an example of a document summary, in accordance with some embodiments;

FIG. 14 illustrates another example of a document summary, in accordance with some embodiments;

FIG. 15 illustrates, in a flowchart, a method of evaluating a ML pipeline performance, in accordance with some embodiments;

FIG. 16 illustrates, in a graph, an example of a ground truth graph, in accordance with some embodiments;

FIG. 17 illustrates, in a graph, an example of a predicted graph, in accordance with some embodiments;

FIG. 18 illustrates, in a flowchart, a method of generating a graph, in accordance with some embodiments;

FIG. 19 illustrates, in a flowchart, another method of generating a graph, in accordance with some embodiments;

FIG. 20 illustrates, in a flowchart, another method of generating a graph, in accordance with some embodiments;

FIG. 21 is a schematic diagram of a computing device such as a server;

FIG. 22 illustrates, in a high level diagram, an example of a pipeline from document and/or image input to the type output, in accordance with some embodiments;

FIG. 23 illustrates a method of preprocessing the documents and/or images, in accordance with some embodiments;

FIG. 24 illustrates an example of classification, in accordance with some embodiments;

FIG. 25 illustrates an example of a transformer, in accordance with some embodiments;

FIG. 26 illustrates another example of a transformer, in accordance with some embodiments;

FIGS. 27A to 27C, which illustrate examples of a merge operation, in accordance with some embodiments;

FIG. 28 illustrates another example of a transformer, in accordance with some embodiments;

FIG. 29 illustrates another example of a classification unit, in accordance with some embodiments;

FIG. 30 illustrates another example of classification unit, in accordance with some embodiments;

FIG. 31 illustrates, in a block-level view, another example of classification unit, in accordance with some embodiments;

FIG. 32 illustrates, in a high-level diagram, an example of an optical character recognition platform, in accordance with some embodiments;

FIG. 33 illustrates an example of an object detection unit, in accordance with some embodiments;

FIG. 34 illustrates an example of an OCR and/or classification unit, in accordance with some embodiments; and

FIG. 35 illustrates an example of a diffusion unit, in accordance with some embodiments.

It is understood that throughout the description and figures, like features are identified by like reference numerals.

### DETAILED DESCRIPTION

Embodiments of methods, systems, and apparatus are described through reference to the drawings.

An automated electronic health record report would allow independent medical examiners (clinical assessors) to perform assessments and efficiently formulate accurate, defensible medical reports. In some embodiments, a system for automating electronic health record reports may be powered by artificial intelligence technologies that consist of classification and clustering algorithms, object character recognition, and advanced heuristics.

Often, a case file may comprise a large number of pages that have been scanned into a portable document format (PDF) or other format. The present disclosure discusses ways to convert a scanned file into an organized format. While files maybe scanned into formats other than PDF, the PDF format will be used in the description herein for ease of presentation. It should be understood that the teachings herein may apply to other document formats.

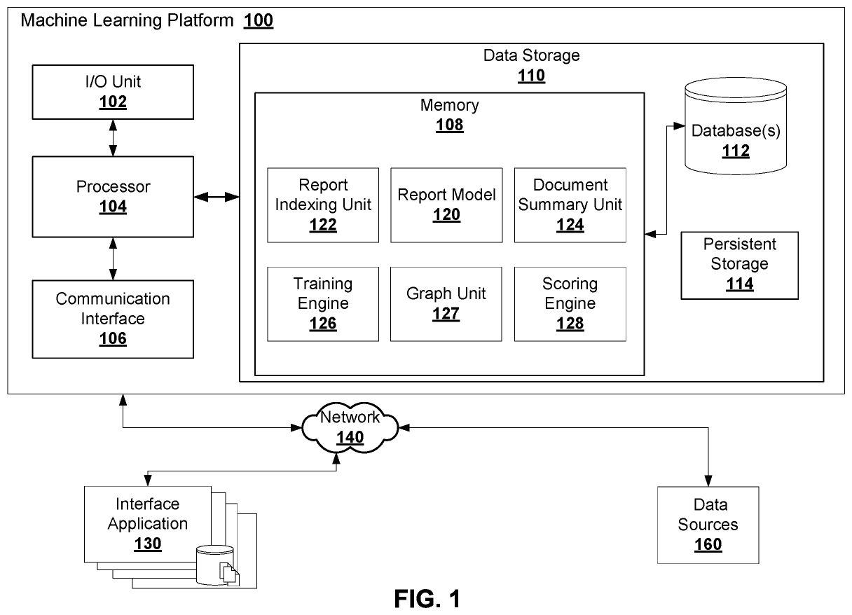

FIG. 1 illustrates, in a schematic diagram, an example of an automated medical report system platform 100, in accordance with some embodiments. The platform 100 may include an electronic device connected to an interface application 130 and external data sources 160 via a network 140 (or multiple networks). The platform 100 can implement aspects of the processes described herein for indexing reports, generating individual document summaries, training a machine learning model for report indexing and summarization, using the model to generate the report indexing and document summaries, and scoring report indexes and summaries.

The platform 100 may include at least one processor 104 and a memory 108 storing machine executable instructions to configure the at least one processor 104 to receive data in form of documents (from e.g., data sources 160). The at least one processor 104 can receive a trained neural network and/or can train a neural network using a machine learning engine 126. The platform 100 can include an I/O Unit 102, communication interface 106, and data storage 110. The at least one processor 104 can execute instructions in memory 108 to implement aspects of processes described herein.

The platform 100 may be implemented on an electronic device and can include an I/O unit 102, the at least one processor 104, a communication interface 106, and a data storage 110. The platform 100 can connect with one or more interface devices 130 or data sources 160. This connection may be over a network 140 (or multiple networks). The platform 100 may receive and transmit data from one or more of these via I/O unit 102. When data is received, I/O unit 102 transmits the data to processor 104.

The I/O unit 102 can enable the platform 100 to interconnect with one or more input devices, such as a keyboard, mouse, camera, touch screen and a microphone, and/or with one or more output devices such as a display screen and a speaker.

The at least one processor 104 can be, for example, any type of general-purpose microprocessor or microcontroller, a digital signal processing (DSP) processor, an integrated circuit, a field programmable gate array (FPGA), a reconfigurable processor, or any combination thereof.

The data storage 110 can include memory 108, database(s) 112 and persistent storage 114. Memory 108 may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. Data storage devices 110 can include memory 108, databases 112 (e.g., graph database), and persistent storage 114.

The communication interface 106 can enable the platform 100 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these.

The platform 100 can be operable to register and authenticate users (using a login, unique identifier, and password for example) prior to providing access to applications, a local network, network resources, other networks and network security devices. The platform 100 can connect to different machines or entities.

The data storage 110 may be configured to store information associated with or created by the platform 100. Storage 110 and/or persistent storage 114 may be provided using various types of storage technologies, such as solid state drives, hard disk drives, flash memory, and may be stored in various formats, such as relational databases, non-relational databases, flat files, spreadsheets, extended markup files, etc.

The memory 108 may include a report model 120, report indexing unit 122, a document summary unit 124, a machine learning engine 126, a graph unit 127, and a scoring engine 128. In some embodiments, the graph unit 127 may be included in the scoring engine 128. These units 122, 124, 126, 127, 128 will be described in more detail below.

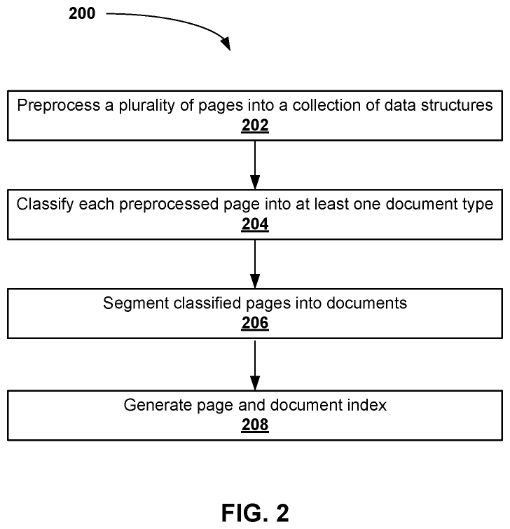

FIG. 2 illustrates, in a flowchart, an example of a method of generating an index of a document 200, in accordance with some embodiments. The method 200 may be performed by the report indexing unit 122. The method 200 comprises preprocessing a plurality of pages into a collection of data structures 202. Each data structure may comprise a representation of data for a page of the plurality of pages. The representation may comprise at least one region on the page. Next, the method 200 classifies each preprocessed page into at least one document type 204. Next groups of classified pages are segmented into documents 206. Next, a page and document index are generated for the plurality of pages based on the classified pages and documents 208. Other steps may be added to the method 200.

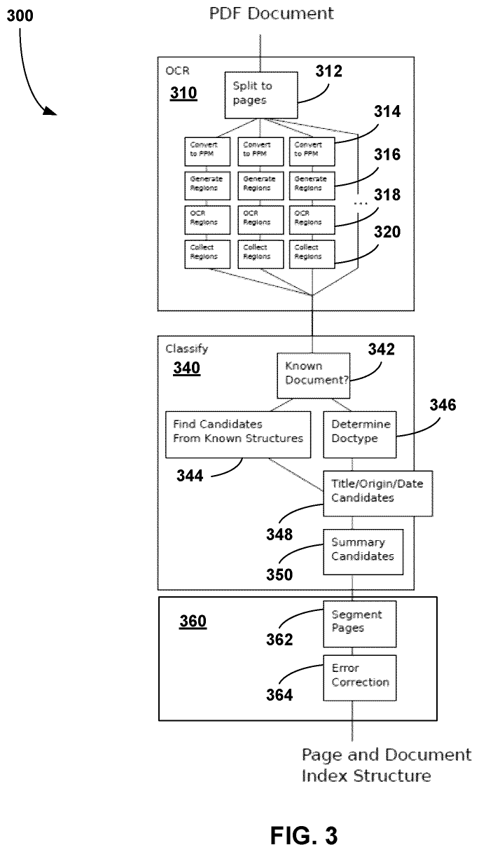

FIG. 3 illustrates, in a flowchart, another example of generating an index of a document 300, in accordance with some embodiments. The method 300 can be seen as involving three main steps: pre-processing 310, classification 340, and report generation 360.

### Preprocessing 310

In some embodiments, predictors are identified and established based on a body of knowledge, such as a plurality of document identifiers that identify official medical record types for different jurisdictions. Which document type to assign to a page may be based off of the document/report model 120. The terms document model and report model are used interchangeably throughout this disclosure. The document model 120 may comprise classification, document index generation and document summary generation. In some embodiments, the document model 120 may comprise/store a document segmentation model, a document type classification model, an attribute (e.g., date, title and facility/origin) extraction model and/or other models. The document model 120 will be further described below.

In some embodiments, complex medical subject matter may be identified using advanced heuristics involving such predictors and/or detection of portions of documents. Is should be noted that a heuristic is a simple decision strategy that ignores part of the available information within the medical record and focuses on some of the relevant predictors. In some embodiments, heuristics may be designed using descriptive, ecological rationality, and practical application parameters. For example, descriptive heuristics may identify what clinicians, case managers, and other stakeholders use to make decisions when conducting an independent medical evaluation. Ecological heuristics may be interrelated with descriptive heuristics, and deal with ecological rationality. For example, to what environmental structures is a given heuristic adapted (i.e., in which environments it performs well, and in which it does not). Practical applications parameters as a heuristic identifies how the study of people's repertoire of heuristics and their fit to environmental structures aid decision making.

In some embodiments, these heuristics may be used in a model 120 that uses predictors for optical character recognition (OCR) applications in any jurisdiction or country conducting medical legal practice. A process using OCR may be used that breaks down a record/document by form. A form may be defined as the sum of all parts of the document's visual shape and configuration. In some embodiments, a series of processes allow for the consolidation of medical knowledge into a reusable tool: identification process, search process, stopping process, decision process, and assignment process.

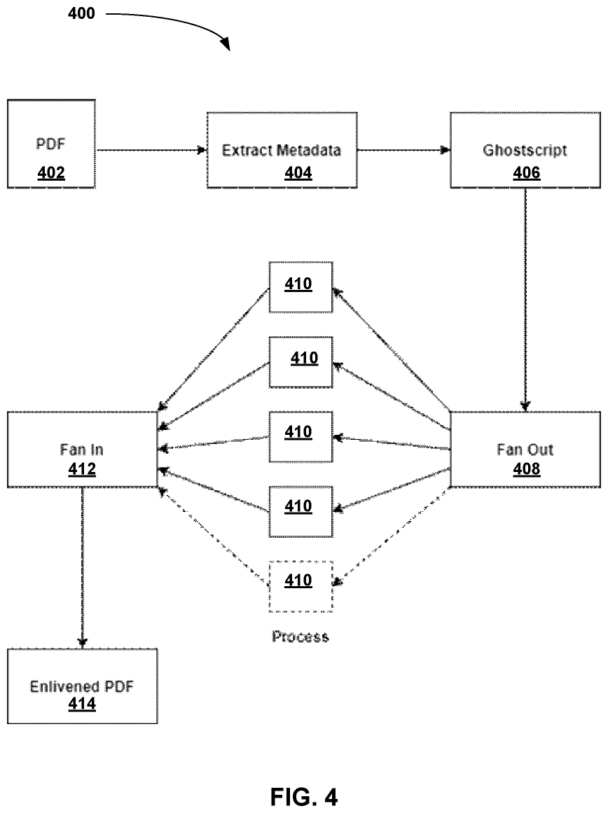

In some embodiments, documents (e.g., PDF documents or other documents) may preprocessed such that content (e.g., text, images, or other content) is extracted and corrected, a search index is built, and the original imaged-PDF is now electronically searchable. FIG. 4 illustrates, in a process flow diagram, an example of a method of preprocessing 400 a PDF document, in accordance with some embodiments. A PDF document 402 is an input which may be “live” or it may contain bitmap images of text that need to be converted to text using OCR. Metadata may be extracted 404 from the PDF document 402. For example, the bookmark and form data may be extracted 404 from the PDF 402. In some embodiments, the extracted data may be saved for future reference. Next, the PDF 402 may be passed through a rendering (such as, for example, ‘Ghostscript’ or any utility function or post-script language interpreter) function 406, to minimize its file size and reduce the resolution of any bitmaps that might be inside. This will allow for the PDF to be displayed more easily in a browser context. Next, the PDF 402 is divided into smaller “chunks” (i.e, Fan Out 408), each of which can be processed in parallel. This is useful for larger files, which will be processed much more quickly this way than working on the entire file at once. Each PDF chunk is enlivened through a separate process 410. For example, this process 410 may involve using a conversion tool such as ‘OCRmyPDF’ to OCR any bitmaps present and embed the result into the PDF chunk. Once all the chunks have been processed, they may be stitched back together (i.e., Fan In 412) in order to provide the output. The output of this process is a fully live, (i.e., enlivened) PDF 414 (rather than a potentially live one). It should be note that an enlivened PDF is a PDF where text and its associated bounding box has been added so that the PDF is searchable.

In some embodiments, an identification process identifies predictors. The system may be configured to receive predictor values (or predictors) that may be assigned to pertinent data points in the document based on location, quadrant, area, and region. In some embodiments, the selection of predictors may be completed by clinical professionals based on experience, user need, medical opinion, and medical body of knowledge. In some embodiments, predictors may be determined and known document patterns and context of pages.

In some embodiments, a search process may involve searching a document for predictors and/or known patterns. For known document types, a specific region may be scanned. For unknown document types, all regions of the document may be scanned to detect the predictors and/or known patterns; such scanning may be performed in the order of region importance based on machine learning prediction results for potential document type categories.

In some embodiments, a stopping process may terminate a search as soon as a predictor variable can identify a label with a sufficient degree of confidence.

In some embodiments, a decision process may classify a document according to the located predictor variable.

In some embodiments, in an assignment process, predictors are given a weight based on importance.

With knowing what to look for (predictors), how to look for it (heuristic), and how to score it by relevance and application, classification algorithms can then accurately identify key pieces of medical information that is relevant to a medical legal user.

Referring back to FIG. 3, classification 340 of a specific form may begin with the OCR 310 of each page to identify specific regions within each page to maximize the identification of certain forms. Forms are the visible shape or configuration of the medical record by page. Typically, forms comprise the following sub regions: a top third region, a middle third region, a bottom third region, a top quadrant region, a bottom 15% region, a bottom right hand corner region, a top right hand corner region, and a full page region. Scanning each sub region provides a better understanding of the medical document and what is to be extracted for the clustering algorithm. The output of this ORC 310 step provides texts of these regions to be processed. The types of data that are used are identifiable and each form can be standardized to allow for accurate production of the existing output on a reoccurring basis. The topology and other features of standardized forms may be included in the document model 120. I.e., the typical regions and layout found on a standardized form may comprise the topology of the standardized form.

The OCR step 310 comprises preprocessing a plurality of pages into a collection of data structures where each data structure may comprise a representation of data for a page of the plurality of pages. The presentation may comprise at least one region on the page. In some embodiments, the OCR 310 step comprises separating a received document (or group of documents comprising a file) into separate pages 312 (shown as “Split to pages” 312). Each page may then be converted to a bitmap file format 314 (shown as “Convert to PPM” 314) (such as a greyscale bitmap, a portable pixmap format (PPM) or any other bitmap format). Regions of interest may also be determined (i.e., generated or identified) on each page 316 to be scanned (shown as “Generate Regions” 316). For example, the system may look at all possible regions on a page and determine if an indicator is present in a subset of the regions. The subset of regions that include an indicator may comprise a signature of the type of form to which the page is a member.

The regions may then be converted into machine-encoded text (e.g., scanned using OCR) 318 (shown as “OCR Regions” 318). The regions and corresponding content (e.g., text, image, other content) may be collected 320 for each page into a data structure for that page (shown as “Collect Regions” 320). In some embodiments, the structure of data for each page represents a mapping of region to content (e.g., text, image, etc.) for each page. Each page data structure may then be merged together (e.g., concatenated, vectored, or formed into an ordered data structure) to form a collection of data structures. It should be noted that steps 314 to 320 may be performed in sequence or in parallel for each page.

### Classification 340

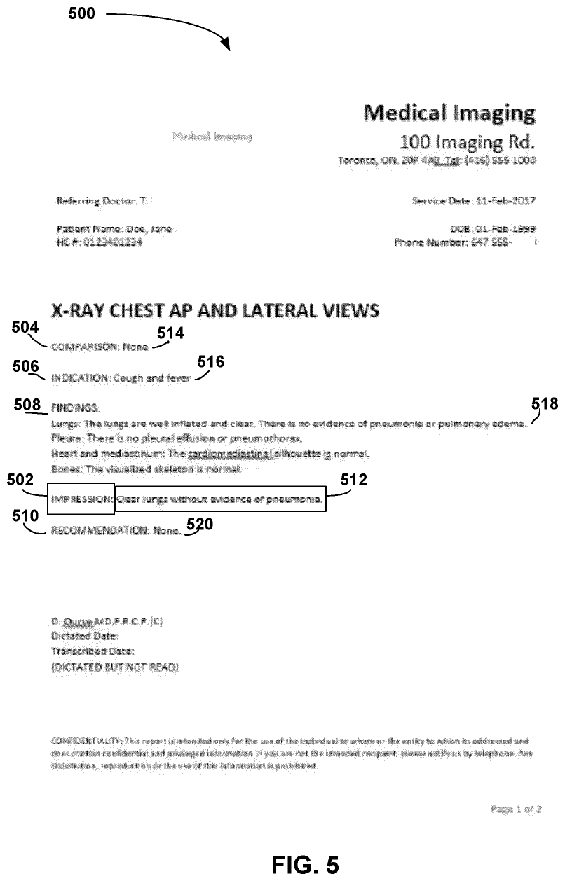

The collection of data structures generated as the output to the OCR/pre-processing step 310 may be fed as input to a classification process 340. The classification process 340 involves the classification of a specific region by a candidate for type. If the document is of a known type 342, then candidates from known structures are located 344. For example, each page is compared with known characteristics of known document types in the model 120. Otherwise 342, the document type is to be determined 346. For example, a feed forward neural network may be trained (using machine learning engine 126) on label corpus of document types to page contents. In some embodiments, a multi-layered feed forward neural network may be used to determine the most likely document type (docType). In some embodiments, the average of word to vector (word2vec) encodings of all the words in a page may be used as input, and the network outputs the most likely docType. In some embodiments, a bidirectional encoder representations from transformers (BERT) language model may be used for the classification. It should be noted that the neural network may be updated automatically based on error correction 364. For example, parameters in the BERT and/or generative pretraining transformer 2 (GPT-2) algorithms may be fine-tuned with customized datasets and customized parameters. This will improve performance. Summarization of documents using such language models may be controlled with a weighted customized word lists and patterns. For example, more weight may be give to words or phrases such as ‘summary’, ‘in summary’, ‘conclusion’, ‘in conclusion’, etc. Patterns may include placement of structure or fragments of text and/or images (or other content) that follow or accompany the words or phrases. For example, FIG. 5 illustrates, in a screenshot, an example of a portion of a PDF page 500 in a PDF document 402, in accordance with some embodiments. The page 500 includes a word ‘IMPRESSION:’ 502 followed by a pattern of content 504 that represents a diagnosis or impression. In this example, the impression is “Clear lungs without evidence of pneumonia.” However, it should be understood that any other diagnosis or impression may be found. It should also be noted that content pattern 512 (e.g., text and/or images and/or other content) does not have to be next to the words 502. The content pattern 512 can be anywhere that is “predictable” in that there is a known pattern for a document type when that word 502 is found, such that the location of the relevant text and/or images are known/predictable. Other examples of words that may be part of a word list in this example include “COMPARISON:” 504, “INDICATION:” 506, “FINDINGS:” 508 and “RECOMMENDATION:” 510, each having a corresponding content pattern 514, 516, 518 and 520.

Candidates (from the document model 120) may comprise headers, document types, summary blocks, origins (people and facility), dates, and page information/identifiers. These candidates are identified and categorized by a page classifier in conjunction with an attribute prediction unit 348. For example, the region data that was received is traversed to select the candidates for each category and assign a candidate score. In some embodiments, a candidate score is a collection of metrics according to clinical expertise. For example, given a block of content, how likely this block of content is what is being searched for is determined. This analysis will provide a title score, a date score, etc. The items that are most likely will be observed in each category. The title/origin/date/etc. candidate items are scored then sorted according to score into a summary 350. Once the candidate items are scored, a key value structure is determined and passed to the clustering step 360 using clustering algorithms. In some embodiments, the structure passed from the classification step 340 to the clustering step 360 comprises a sequence of key/value maps that includes an ‘index’ value (e.g., the integer index of the given page in the original document), one or more ‘regions’ values (e.g., the region data extracted via OCR process 318), and ‘doc_type’ (or ‘docType’), ‘title’, ‘page’, ‘date’, ‘origin’ and ‘summary’ values (e.g., ordered sets of candidates of each property descending by correctness likelihood).

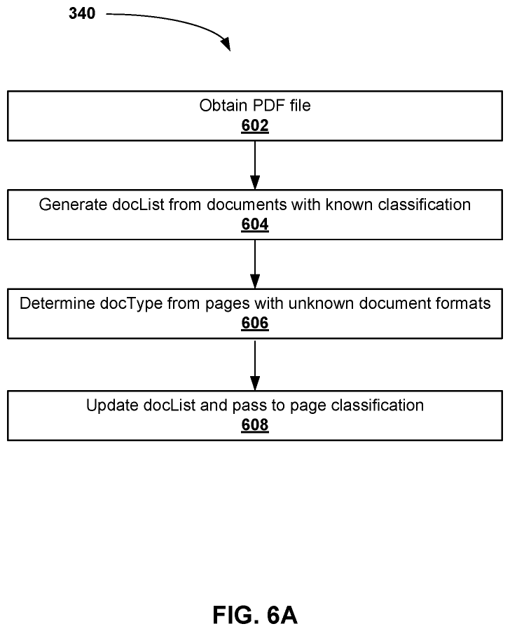

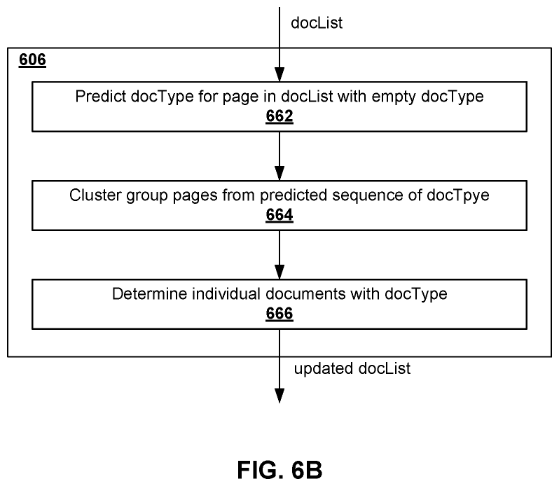

FIG. 6A illustrates, in a flowchart, another example of a method for classifying pages 340, in accordance with some embodiments. The method 340 begins with obtaining a PDF file 602. For a given PDF file, a known_docs classifier processes and extracts all pages with known document formats 344 (from document model 120), and from these pages further extracts their meta information (e.g., title, origin (e.g., institution, clinic, provider, facility, etc.), author, date, summary, etc. 348, 350). A docList is generated 604 with pages that are extracted with meta information and with pages that are not extracted (i.e., pages that did not match with a known document format in the document model 120). The docList is passed to a docType classifier where pages with empty docType information are processed 606. A docType from pages with unknown document formats is obtained, and the docList is updated and passed 608 to page classification. Page classification will predict candidates for meta information (e.g., title, origin, author, date, summary, etc. 348, 350) for pages of unknown document types.

FIG. 6B illustrates, in a flowchart, an example of a method for determining a docType from pages with unknown document formats 346, 606, in accordance with some embodiments. The method 346, 606 begins with predicting 662 a docType for each page in docList with empty docType. In some embodiments, predicting involves generating candidate meta information 348, 350, using the trained model 120 for key words and patterns that are likely for a document type (docType). Typically, the document type with the highest likelihood is used. In some embodiments, the machine learning engine ingests pages in its neural network, outputs the probabilities of all possible document types, and selects the docType with the highest probability/likelihood as the docType of the pages. After processing all pages, a sequence of docTypes with page number is generated. If some docType is predicted for a page, then this page is labeled as the first page of that document. If no docType is obtained, then the page is not the first page. From the predicted sequence of docTypes group pages are clustered 664 into different documents with docTypes. In some embodiments, clustering 664 involves grouping similar pages (based on a vector which will be further described below) into one document. Thus, individual documents with docType are determined 666.

For example, suppose that the predicted sequences of docTypes is:

(5,report), (6,none), (7,none), (8,assessment), (9,none), (10,image), (11,none), (12,none).

This predicted sequence represents that patterns were found on “page 5” that suggest that the most likely docType for “page 5” is a report, patterns were found on “page 8” that suggest that the most likely docType for “page 8” is an assessment, and patterns were found on “page 10” that suggest that the most likely docType for “page 10” is an image. In this example, no patterns were found for pages 6-7, 9 or 11-12. In some embodiments, a minimum threshold of likelihood (e.g., 50% or another percentage) may be used to distinguish between a pattern likelihood worthy of labelling a docType and a pattern likelihood too low to label a docType for a page.

Pages with “none” (i.e., where no docType has been predicted thus far) that follow a page having a predicted docType can be inferred to be of that same docType. Thus, for pages 5-12, it can be concluded that pages 5-7 is a report, pages 8-9 is an assessment, and pages 10-12 is an image. In some embodiments, pages 5 to 7 may be encoded to represent a document, pages 8 and 9 encoded to represent an assessment, and pages 10 to 12 encoded to represent an image. The three individual documents may then be processed separately by the page classifier 348 to predict the missing meta information.

### Clustering 360

Referring back to FIG. 3, pages may be segmented (i.e., grouped into document types) 362. Using the raw data (e.g., title, author/origin, date, etc. obtained in the classification 340), list of candidates and collected candidate summaries, the pages are analyzed and associated with each other where possible. For example, pages may be grouped together based on similar document types, similar titles, sequential page numbers located at a same region, etc. It has been observed that the strongest associations involve document title, groups, and pages. For example, some pages have recorded page numbers (such as “1 of 3” or “4 of 7” or “1/12”). If contiguous pages are located that all report the same total page count, and no conflicting page numbers, they are likely to be grouped (for instance, if pages are located in sequence that are labelled as “1 of 5”, “2 of 5”, “3 of 5”, “4 of 5”, “5 of 5”, then they are very likely to constitute a group).

Once pages are segmented 362, an initial grouping of characteristics by page and by document is provided. Error correction 364 may take place to backfill missing data from the previous step (e.g., a missing page number). Errors are identified and adjusted by a clustering algorithm. In some embodiment, based on the information in the key value structure, groups of pages that are together (diagnostics, etc.), groups of relevant content based on scoring, and groups of relevant forms can all be identified.

For example, there may be 3 pages in row and perhaps the middle page number is mangled (e.g., fuzzy scan, page out of order, unexpected or unreadable page number). An inference may be created based on what is missing. Pages to which no grouping was assigned may be analyzed. In some embodiments, there is a manual tagging system (using supervised learning) that can assign attributes such as title, author, date, etc. to documents.

The machine will compare the BERT or Word2Vec generated vectors of mangled page with other pages' vectors, and group this page into the group with most relevance. Also, page number could be used for assistance when a group misses a page. If metadata is missing from a page, then the machine can extract the information (such as author, date, etc.) using natural language process tools such as name-entity recognition. A confidence score may then be calculated and by the model and assigned to each metadata according to its page number in the group.

If a title, page number, or any other characteristic is missing for an ungrouped page, but all other characteristics are the same for a grouping, then there is a confidence score that can be assigned by the model to that page to be inserted/added to the grouping. Pages with low confidence may be trimmed from a grouping for manual analysis. Stronger inferences may be obtained with “cleaned” data sets. For example, pages with low confidence may be reviewed for higher accuracy. In some embodiments, a threshold confidence level may be defined for each class/category of document having a low confidence score. Such results may be used to train the model 120.

Once groups of data are smoothed out and organize, the data may be fed into a document list generation function to output a page and document index structure (e.g., docList). In some embodiments, document list generation comprises i) completing a candidate list and indexing the candidates, ii) generating a document structure/outline based on the likeliest page, date, title, and origin, iii) creating a list generator which feeds off of the clustering algorithm and itemizes a table of contents (i.e., after clustering all pages into documents and extracting all meta information for these documents, then these meta information and page ranges of documents can be listed in a table of contents), and iv) taking the table of contents and converting it into a useable document format for the user (i.e., adding the generated index/table of contents to the original PDF file).

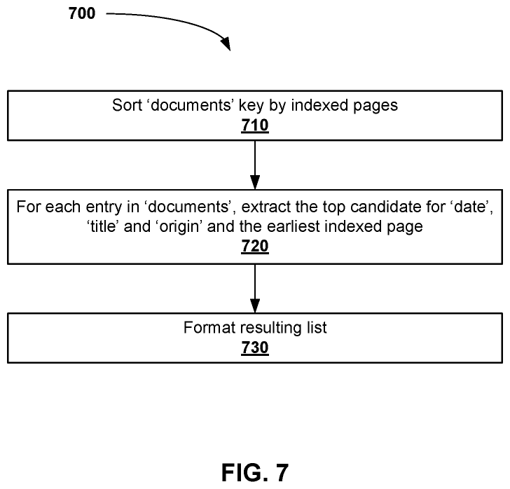

FIG. 7 illustrates, in a flowchart, an example of a method of generating an index (or a table of contents) 700 from the output of the classification component, in accordance with some embodiments. The method comprises sorting the ‘documents’ key by indexed pages 710, extracting the top candidate for ‘date’, ‘title’ and ‘origin’, and the earliest indexed page for each entry in ‘documents’ 720, and formatting the resulting list 730 (for example as a PDF, possibly with hyperlinks to specified page indices). Other steps may be added to the method 700.

In some embodiments, the system and methods described above use objective criteria to remove an individual's biases allowing the user to reduce error when making a decision. Decision making criteria may be unified across groups of users improving the time spent on the decision-making process. Independent medical evaluation body of knowledge may be leveraged to enhance quality, accuracy, and confidence.

In some embodiments, the document summary unit 124 may comprise a primitive neural-net identifier of the same sort as that used on title/page/date/origin slots. In some embodiments, a natural language generation (NLG)-based summary generator may be used.

In some embodiments, a process for identifying how a medical body of knowledge is synthesized and then applied to a claims process of generating a medical opinion is provided.

In some embodiments, a sequence of how a medical document is mapped and analyzed based on objective process is provided.

In some embodiments, a method for aggregating information, process, and outputs into a single document that is itemized and hyperlinked directly to the medical records is provided.

In some embodiments, an automated report comprises a document listing, and a document review/summary. A detailed summary of the document list may include documents in the individual patient medical record that are identified by document title. In some embodiments, the documents (medical records) are scanned (digitized) and received by the system. These medical records are compiled into one PDF document and can range in size from a few pages (reports) to thousands of pages. The aggregated medical document PDF is uploaded into an OCR system. The OCR system uses a model to map specific parts of the document. The document is mapped and key features of that document are flagged and then aggregated into a line itemized list of pertinent documents. The document list is then hyperlinked directly to the specific page within the document for easy reference. The list can be shared with other users.

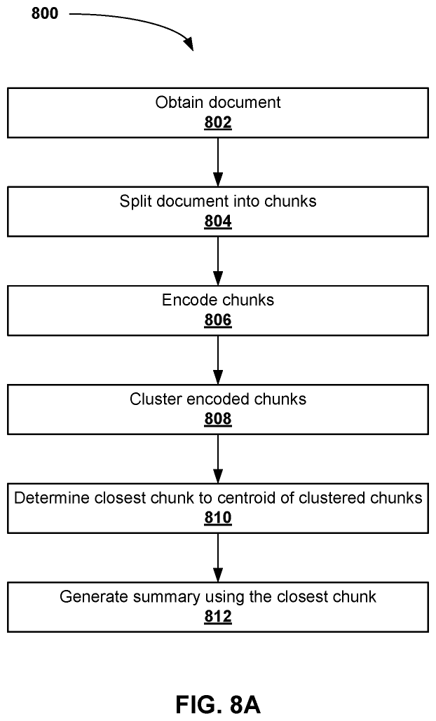

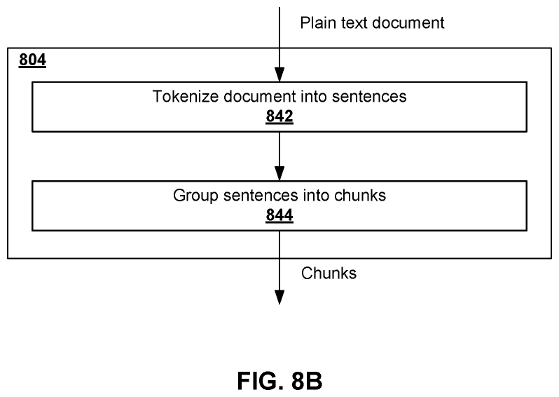

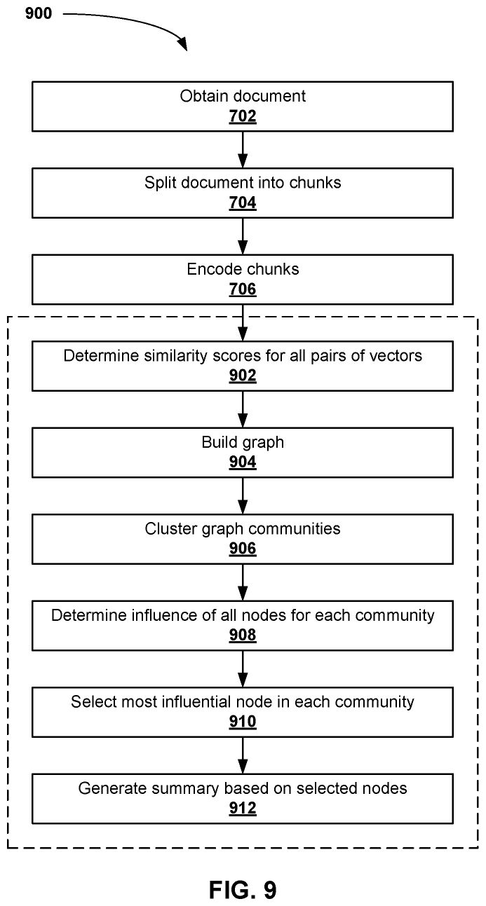

Once a set of PDF pages are categories into a list of documents, each document may be summarized. There are different approaches to summarizing a given document, including extractive summarization and generative summarization. Extractive summarization is different from generative summarization. Extractive summarization will extract import sentences and paragraphs from a given document, where no new sentences are generated. In contrast, generative summarization will generate new sentences and paragraphs as the summary of the document by fully understanding the content of the document. Extractive methods will now be discussed in more detail, including K-means clustering based summarization (see FIG. 8A), and relational graph based summarization (see FIG. 9).

Clustering may be applied for extractive summarization by finding the most important sentences or chunks from the document. In some embodiments, BERT-based sentence vectors may be used. Graph-based clustering may be used to determine similarities or relations between BERT-based vectors and encoded sentences or “chunks” of content. In some embodiments, BERT-based vectors may be used to assist with computing the graph community and extracting the most important sentences and chunks with a graph algorithm (e.g., PageRank).

Generative summaries may be created using a graph-based neural network trained over a dataset. Summaries such as GPT-2 may be generated. It should be noted that other GPT models may be use, e.g., GPT-3.

FIG. 8A illustrates, in a flowchart, an example of a method of summarizing a document 800, in accordance with some embodiments. The method 800 may be performed by the document summary unit 124. The method 800 obtaining a document 802, dividing or splitting the document into groupings of content (i.e., “chunks”) 804, encoding the chunks into a natural language processing format (e.g., word2vec or BERT-based vectors) into the chunks 806, clustering the encoded chunks 808 into groupings based on their encodings, determining the most central points (e.g., closest chunk to the centroid of the clustered chunks) 810 of the clustered chunks, and generating a summary 812 for the document based on the most central points (e.g., closest chunk) Other steps may be added to the method 800. It should be noted that a “chunk” comprises a group of content such as, for example, a group of sentences and/or fragments, whether continuous or not in the original document.

The method 800 will now be described in more detail. In some embodiments, K-means clustering may be used in the method 800. For example, a plain text document may be received as input 802 (which could be the OCR output from a PDF file, or image file). Next, the document can be divided or split into chunks.

FIG. 8B illustrates, in a flowchart, a method of dividing a document into chunks 804, in accordance with some embodiments. Suppose the atom of summarization is a sentence. With natural language processing tools, the plain text document 802 may be tokenized 842 into sentences, and chunks of content are built 844 upon these sentences 804. There are many ways for the system to generate chunks. One way is to tokenize the document into sentences or fragments, and group the number of sentences or fragments by their indices. Another way to group a number of sentences and/or fragments by their correlation/relation/relevance (e.g., two or more fragments or sentences comprise a chunk). It should be noted that a different number of fragments and/or sentences can comprise a chunk. In some embodiments, differently sized chunks may be defined for different document types. It should be noted that a chunk may comprise one or several sentences and fragments (or other types of content) whether or not they are continuous or in order from the original document. Other steps may be added to the method 804.

Referring back to FIG. 8A, BERT or other vectorizing or natural language processing methods may be applied to each chunk 806. Each chunk will be converted into a high dimensional vector. BERT and Word2Vec are two approaches that can convert words and sentences into high dimensional vectors so that mathematical computation can be applied to the words and sentences. For example, the system may generate a vocabulary for the entire context (based on trained model), and input the index of all words of sentences/chunks in the vocabulary to a BERT/Word2Vec based neural network, and output a high dimensional vector, which is the vector representation of the chunk. The dimension of the vector may be predefined by selecting the best tradeoff between speed and performance.

In some embodiments, a vocabulary may comprise a fixed (not-necessarily alphabetical) order of words. A location may comprise a binary vector of a word. If a chunk is defined to be (X-ray, no fracture seen, inconclusive), and vocabulary includes the words “X-ray”, “fracture”, and “inconclusive”, then the corresponding vector for the chunk would be the average of the binary locations for “X-Ray”, “fracture”, and “inconclusive” in the vocabulary.

In some embodiments, the neural network may input chunks and generate vectors. Using K-means clustering (or other clustering methods), the set of high dimensional vectors may be clustered into different clusters 808. I.e., by looking at the distance between vectors of chunks, the algorithm may dynamically adjust groups and their centroid to stabilize clusters until an overall minimum average distance is achieved. The distance between high-dimensional vectors will determine the vectors that form part of that cluster. N clusters may be predefined where N is the length of the summary for the document. For each cluster generated in step 808, the vector that is closest to the centroid of the cluster 810 is used. In some embodiments, a cosine distance may be calculated to determine the distance between vectors. The closest N vectors could also be used rather than just the closest vector to the center of the centroid. It should be noted that N could be preset by a user, and that there can be a different value for N for different docLists. If a longer summary is desired, then a larger N may be chosen. By mapping the closest vectors back to their corresponding chunk, those chunks may be joined to generate the summary 812 of the document.

FIG. 9 illustrates, in a flowchart, another method of summarizing a document 900, in accordance with some embodiments. The first three steps 802, 804 and 806 of this approach are the same as that of the method described in FIG. 8A (for which K-means clustering is used in some embodiments). After obtaining the vectors for the chunks 806, a similarity calculation 902 may be used to determine or compute all similarity scores between all pairs of vectors (e.g., using a cosine metric). For each pair of vectors, if their similarity score is greater than a predefined threshold, then the two vectors are connected. Otherwise, there is no connection between those two vectors. In this way, a graph is built 904 with vectors as the nodes, and connections as the edges. Clustering over the graph 906, a set of subgraphs called communities are generated where within each community all nodes are closely connected. In some embodiments, the nodes are considered to be closely connected when they have high relevance scores and more connections. The higher the relevance score between sentences, the more likely those sentences are connected. For each community, influence of all nodes may be determined 908. The most influential node may be defined as the node that has the most number of connections with all other nodes within the community, and these connection have high similarity scores as well. Next, the nodes of the community may be sorted by influence, the node with the most influence 910 may be selected to represent that community. The selected or chosen nodes or vectors may be mapped back to their corresponding chunks of content. The corresponding chunks of content may then be joined to form the summary of the document 912. Other steps may be added to the method 900.

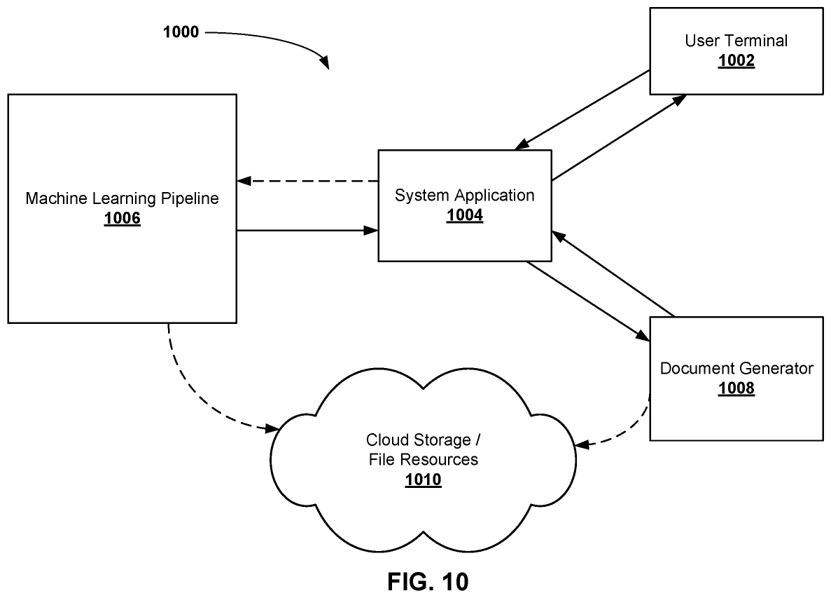

FIG. 10 illustrates, in a schematic, an example of a system environment 1000, in accordance with some embodiments. The system environment 1000 comprises a user terminal 1002, a system application 1004, a machine learning pipeline 1006, a document generator 1008, and a cloud storage 1010. In some embodiments, the user terminal 1002 does not have direct access to internal services. Such access is granted via system application 1004 calls. The system application 1004 coordinates interaction between the user terminal 1002 and the internal services and resources. Permissions to the file resources/memory storage may be granted to software robots on a per use basis.

In some embodiments, the system application 1004 may be a back-end operation implemented as a Python/Django/Postgres application that acts as the central coordinator between the user and all the other system services. It 1004 also handles authentication (verifying a user's identification) and authorization (determining whether the user can perform an action) to internal resources. All of the system application 1004 resources are protected, which includes issuing the proper credentials to internal robot/automated services.

Some resources that may be created by the system application 1004 include User Accounts, Cases created, and Files uploaded to the Cases. After an authentication process, the frontend (i.e., user terminal 1002) may request the backend (i.e., system application 1004) to create a Case and to upload the Case's associated Files to the system application 1004. In some embodiments, files are not stored on the system application 1004. The cloud storage/file resources 1010 may be a service used to provide cloud-based storage. Permissions are granted to a file's resource based on a per-user basis, and access to resources are white-listed to each client's IP.

Services with which that the system application 1004 communicates include an index engine 122 (responsible for producing an index/summary) and PDF generator (responsible for generating PDFs). In some embodiments, the contents of files are not directly read by the system application 1004 as the system application 1004 is responsible for coordinating between the user terminal 1002 and underlying system machine-learning pipeline 1006 and document generating processes 1008.

As noted above, the BERT language model (e.g., https://arxiv.org/abs/1810.04805) may be used to obtain a vector representation of the candidate strings using a pre-trained language model. The vector representation of the string then passes through a fine-tuned multi-layer classifier a trained to detect titles, summaries, origins, dates, etc.

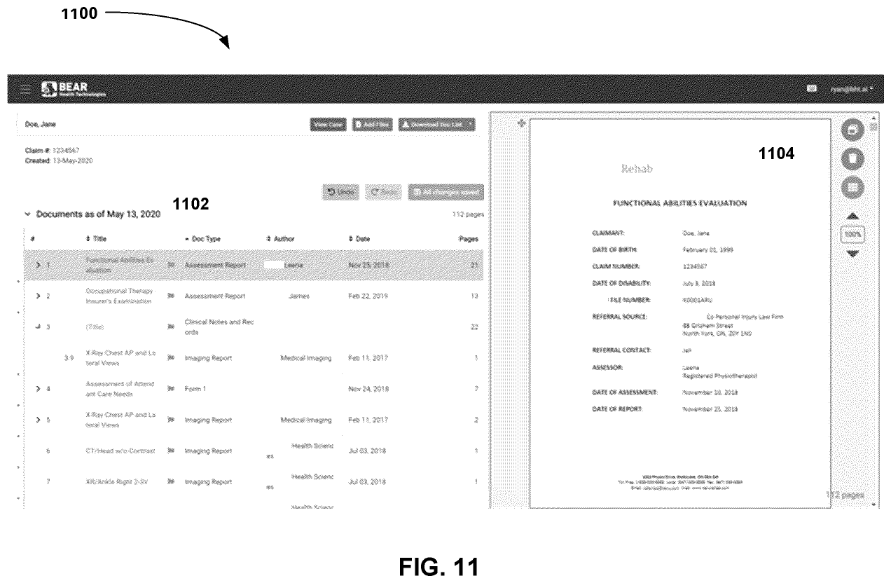

In some embodiments, an index or document list (e.g., docList) may be generated. FIG. 11 illustrates, in a screen shot, an example of an index 1100, in accordance with some embodiments. FIG. 11 shows a listing 1102 of documents. In this example, the documents are listed in a table/spreadsheet format with document number (#), Title, Doc Type, Author, Date and number of Pages as the columns. The first document shown is titled “Functional Abilities Evaluation”, is an “Assessment Report” document type, is Authored by Leena on Nov. 25, 2018 and has 21 pages. It should be noted that different documents of the same or different types, authored by the same or different authors, on the same or different dates, and having the same or different number of pages, may be shown in other instances or examples. In this index 1100, when a row entry in the listing 1102 is selected or highlighted, a preview of that document is shown in a preview pane 1104. As noted above, in this example, the listing 1102 includes columns for index number, title, docType, author, date and number of pages. Other columns in a similar or different order may be produced. The preview pane 1104 shows an image of the first page of a document entitled “Functional Abilities Evaluation” that corresponds to the first item that was selected in the listing 1102.

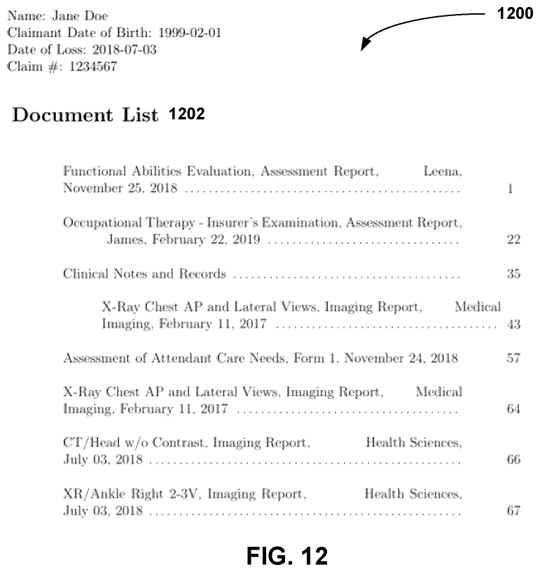

FIG. 12 illustrates another example of an index 1200, in accordance with some embodiments. FIG. 12 shows a listing 1202 (or document list). In this example, the listing 1202 appears as a table of contents where each entry includes a title, docType, author, date and first page number for the listing. For example, the first entry in the table of contents shows “Functional Abilities Evaluation, Assessment Report, Leena, Nov. 25, 2018 . . . 1”. The second entry shows “Occupational Therapy—Insurer's Examination, Assessment Report, James, Feb. 22, 2019 . . . 22”. It should be noted that these are simply example documents that match the documents listed in index 1100.

The index 1100, 1200 may include an automatically generated hyperlinked index with line items corresponding to documents/files uploaded to a case.

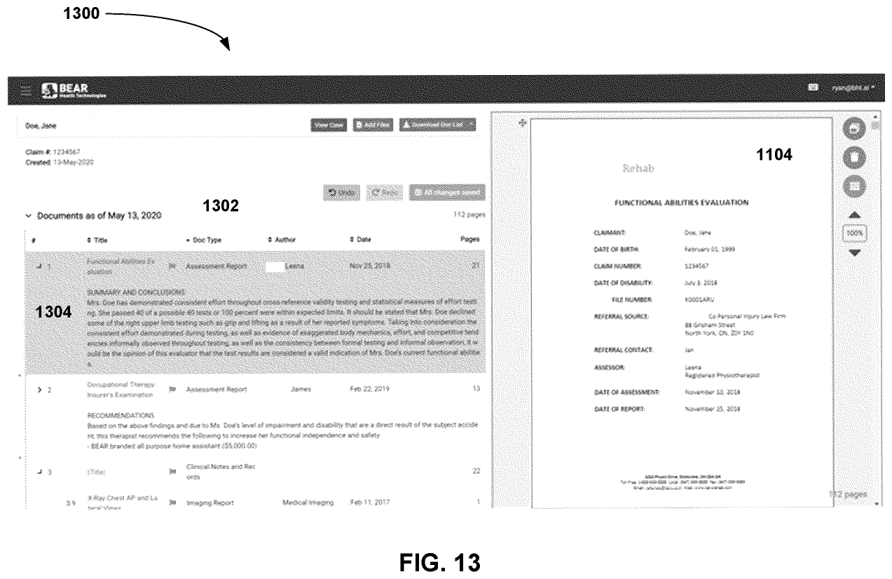

In some embodiments, a summary or document review may be generated. FIG. 13 illustrates, in a screen shot, an example of a document summary 1300, in accordance with some embodiments. FIG. 13 shows a listing 1302 that also includes a summary 1304 row (e.g., Summary and Conclusions, Recommendations, etc.) immediately below a corresponding listing row. This example shows the same table/spreadsheet as in FIG. 11 with an additional row 1304 below each entry that includes the summary. For example, the summary for the first entry states:
SUMMARY AND CONCLUSIONS
Mrs. Doe has demonstrated consistent effort throughout cross-reference validity testing and statistical measures of effort testing. She passed 40 of a possible 40 tests or 100 percent were within expected limits. It should be stated that Mrs. Doe declined some of the right upper limb testing such as grip and lifting as a result of her reported symptoms. Taking into consideration the consistent effort demonstrated during testing, as well as evidence of exaggerated body mechanics, effort, and competitive tendencies informally observed throughout testing, as well as the consistency between formal testing and informal observation, it would be the opinion of this evaluator that the test results are considered a valid indication of Mrs. Doe's current functional abilities.
   It should be noted that the summary 1304 is merely an example and other types of document summaries may be associated with entries. The summary for the second document is entitled “RECOMMENDATIONS”. The same first page view of the selected first document is shown in the window pane 1104.

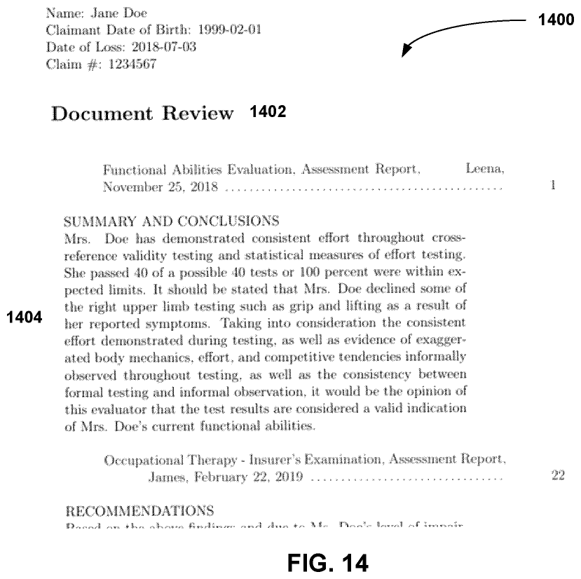

FIG. 14 illustrates another example of a document summary 1400, in accordance with some embodiments. FIG. 14 shows a listing 1402 that also includes a summary content 1404 (e.g., Summary and Conclusions, Recommendations, etc.) immediately below a corresponding a table of contents entry. In this example, the entire “SUMMARY AND CONCLUSIONS” paragraph from FIG. 13 is shown 1404 below the listing of the first document. The “RECOMMENDATIONS” paragraph for the second document is shown beginning below the second entry listing.

Direct summaries may be extracted from documents/files (as described above) and attached to corresponding hyperlinked line items.

In some embodiments, a scoring system may help evaluate a machine learning (ML) model's performance. It is nontrivial to define a good evaluation approach, and even harder for a ML pipeline, where there are many ML models entangled together. An approach to evaluating a ML pipeline's performance will now be described. This approach is based on relational graph building and computation. For known document classification, the scoring system may address how the accuracy affects blocks of content associated with the known document. For document type classification, the scoring system may be associated with accuracy of the classification, and how an incorrect prediction and document separation between blocks of content may affect other indexes (such as, for example, how an incorrect prediction will affect the author, date, etc. for other indexes). Edit distance may be used to compute similarity.

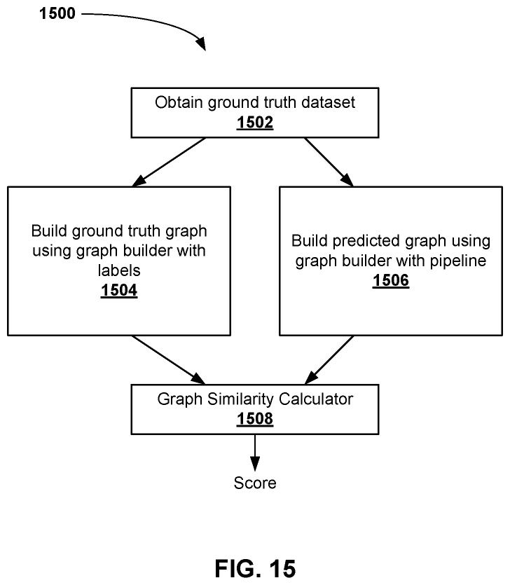

FIG. 15 illustrates, in a flowchart, a method of evaluating an ML pipeline performance 1500, in accordance with some embodiments. The method 1500 may be performed by the scoring engine 128. A ground truth data set is obtained 1500. A ground truth graph 1600 may be built 1504 using a graph builder with labels. A predicted graph 1700 may also be built 1506 using a graph builder with the methods described above. A graph similarity score between the ground truth graph 1600 and the predicted graph 1700 may be determined 1508. Other steps may be added to the method 1500.

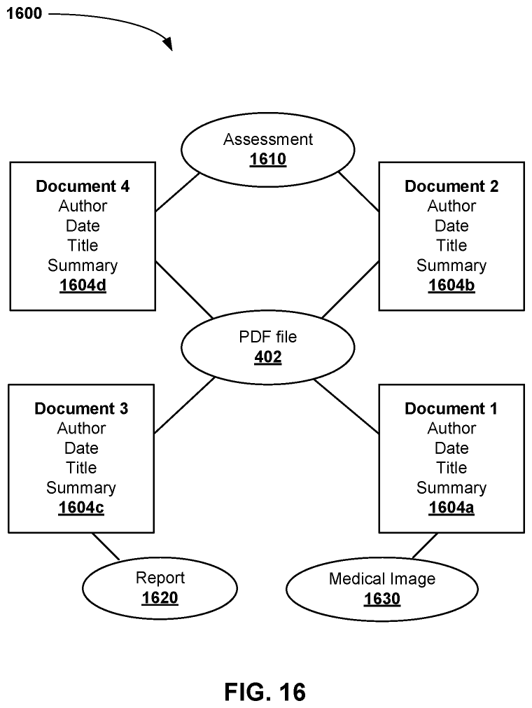

Given a ground truth dataset with manual labels 1502. For each PDF file 1602 and its labels in the dataset, a graph may be built 1504 with nodes as individual documents, types. FIG. 16 illustrates, in a graph, an example of a ground truth graph 1600, in accordance with some embodiments. The PDF file 1602 includes four documents 1604a, 1604b, 1604c, 1604d, with three different doc types (assessment 1610, report 1620 and medical image 1630), and each document has several attributes: author, date, title and summary. It should be noted that other examples of document types may be used.

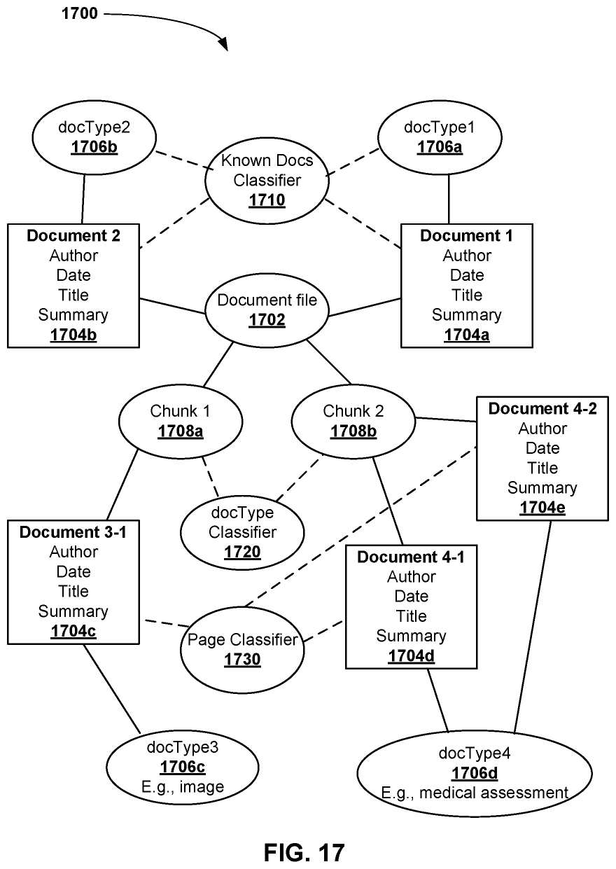

For the same PDF file 1602 in the dataset, the methods described above may be applied on the file to predict the attributes. A predicted graph 1700 may then be built 1506. FIG. 17 illustrates in a graph, an example of a predicted graph 1700, in accordance with some embodiments. First, a known document classifier 1710 may extract 344 all known format files and their attributes. Then, a document type classifier 1720 may split (chunk 1 1708a, chunk 2 1708b) the unclassified pages into separate documents based on their docType 1706a, 1706b, 1706c, 1706d, and then feed these documents into a page classifier 1730 to obtain their predicted attributes.

A graph similarity calculator may be used to determine 1508 the distance or similarity between the ground truth graph 1600 and the predicted graph 1700. For example, a graph edit distance may be determined. In some embodiments, the similarity can be used as a metric to evaluate the machine learning pipeline's performance as compared with the ground truth. If the similarity score is higher than a predefined threshold, then there can be confidence to deploy the ML pipeline into production. Otherwise, the models 120 in the pipeline could be updated and fine-tuned with new dataset(s). Commonly seen unknown document types with low confidence can be hard coded into future version of the system.

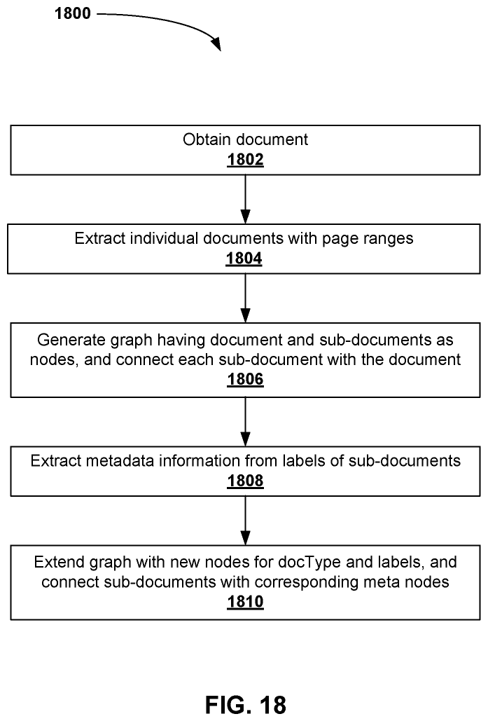

FIG. 18 illustrates, in a flowchart, a method of generating a graph 1800, in accordance with some embodiments. The method 1800 may be performed by the graph unit 127 and/or scoring engine 128. The method 1800 comprises obtaining a document file 1802 (such as, for example, receiving a PDF document 402 having manually inserted or machine-generated labels). Individual documents (i.e., sub-documents) may be extracted 1804 with page ranges. A graph may then be generated 1806 having the original document file and all sub-documents as nodes. Each sub-document may be connected with an edge to the original document file. Next, metadata information may be extracted 1808 from labels (e.g., docType, title, author/origin, date, summary, etc.) of the sub-documents. The graph may be extended 1810 with new nodes for docType and labels for each sub-document. Edges may be added connecting the sub-documents with their corresponding meta information (e.g., docType, title, author/origin, date, summary, etc.). If the obtained document file 1802 was a document having manually inserted labels, then a ground truth graph has been generated. If the obtained document file 1802 was a document having machine-generated labels, then a machine-generated graph has been generated. Other steps may be added to the method 1800.

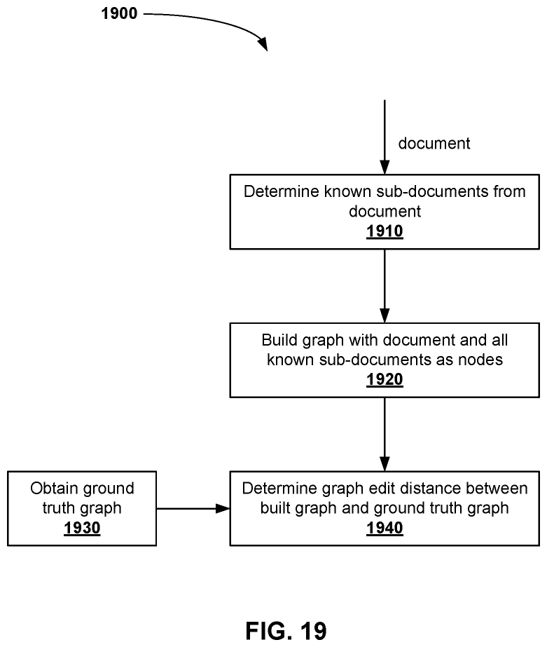

FIG. 19 illustrates, in a flowchart, another method of generating a graph 1900, in accordance with some embodiments. The method 1900 may be performed by the graph unit 127 and/or scoring engine 128. In some embodiments, the machine generated graph can be built on the fly. For example, after a known document classifier processes 1910 the document file 402, a graph can be generated 1806, 1920 that comprises the document file and all known sub-documents as nodes. At this point, the edit distance between this graph and an obtained 1930 ground truth graph (i.e., received, fetched or generated ground truth graph) can be determined 1940 using known techniques such as, for example, Levenshtein distance, Hamming distance, Jaro-Winkler distance, etc. This similarity/distance may be used to evaluate the known document classifier. Other steps may be added to the method 1900.

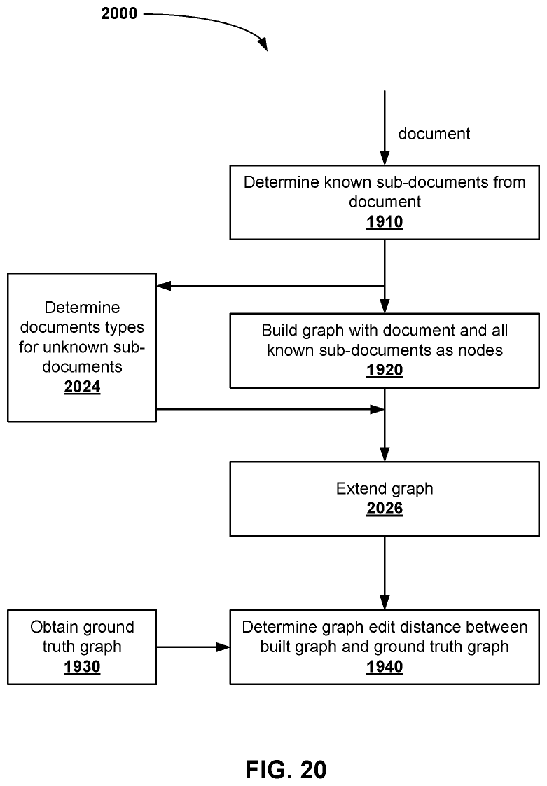

FIG. 20 illustrates, in a flowchart, another method of generating a graph 2000, in accordance with some embodiments. The method 2000 may be performed by the graph unit 127 and/or scoring engine 128. The method 2000 begins with determining the known sub-documents 1910, and generating a graph 1920 comprising the document file and all known sub-documents. After a docType classifier processes 2024 the pages in the document 402 having unknown document types, the graph may be extended 2026 with the additional docTypes and sub-documents determined by the docType classifier. The distance between this updated graph and the obtained 1930 ground truth graph may be determined 1940. This similarity/distance may be used to evaluate the combined performance of known document classifiers and document type classifiers. Once the similarity/distance scores reach a threshold value, then the system is ready to be deployed (i.e., the model 120 has been sufficiently trained). Other steps may be added to the method 2000.

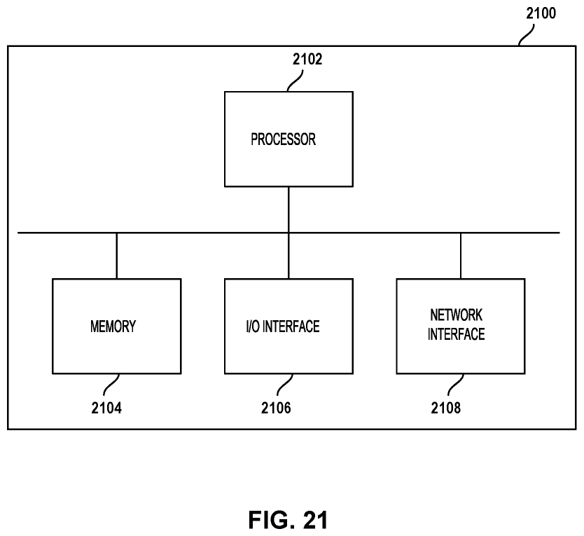

FIG. 21 is a schematic diagram of a computing device 2100 such as a server. As depicted, the computing device includes at least one processor 2102, memory 2104, at least one I/O interface 2106, and at least one network interface 2108.

Processor 2102 may be an Intel or AMD x86 or x64, PowerPC, ARM processor, or the like. Memory 2104 may include a suitable combination of computer memory that is located either internally or externally such as, for example, random-access memory (RAM), read-only memory (ROM), compact disc read-only memory (CDROM).

Each I/O interface 2106 enables computing device 2100 to interconnect with one or more input devices, such as a keyboard, mouse, camera, touch screen and a microphone, or with one or more output devices such as a display screen and a speaker.

Each network interface 2108 enables computing device 2100 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others.

Document and/or Image Type Prediction

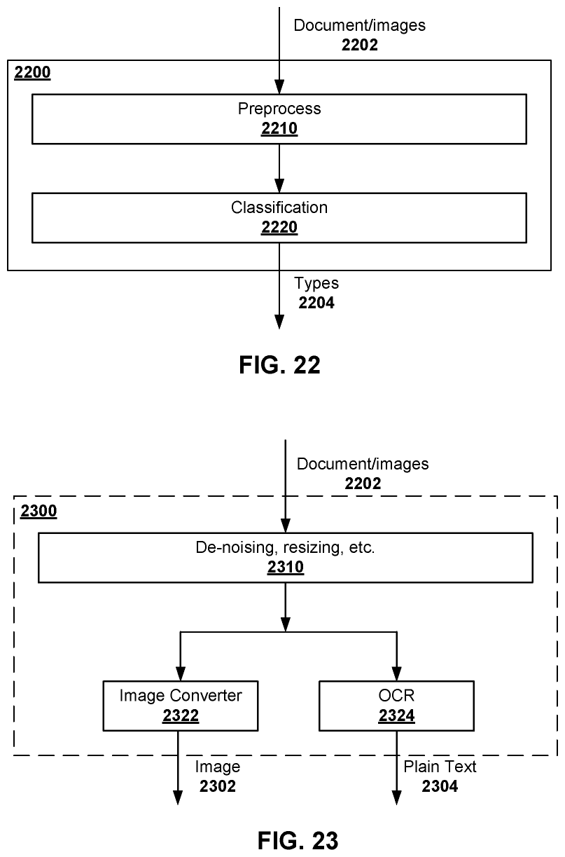

FIG. 22 illustrates, in a high level diagram, an example of a pipeline from document and/or image input 2202 to the type output 2204, in accordance with some embodiments. In some embodiments, the documents could be word documents and PDF, while images could be in PNG, JPG, and/or TIFF format, to name a few. These documents and images will be preprocessed 2210 before being ingested by the classification 2220, predicting the document type or image type 2204.

FIG. 23 illustrates a method 2300 of preprocessing the documents and/or images 2202, in accordance with some embodiments. In some embodiments, the input documents and images will be preprocessed (e.g., normalized, de-noised, cleaned up, converted into greyscale or black-white format, resized, etc.) 2310 to improve the OCR 2324 accuracy of plain text 2304. In some embodiments, normalizing, de-noising and cleaning up may comprise, for example, using a utility to remove background noise such as artifacts or other unwanted markings following an OCR conversion of a document. The cleaned images may be further converted 2322 into another format (such as an image or other type of format, including an enlivened PDF or other types of fillable documents) 2302 for classification. It should be understood that an enlivened PDF (or other types of fillable documents) comprises both an image and plain text with bounding boxes.

Several embodiments will be described for classification 2220.

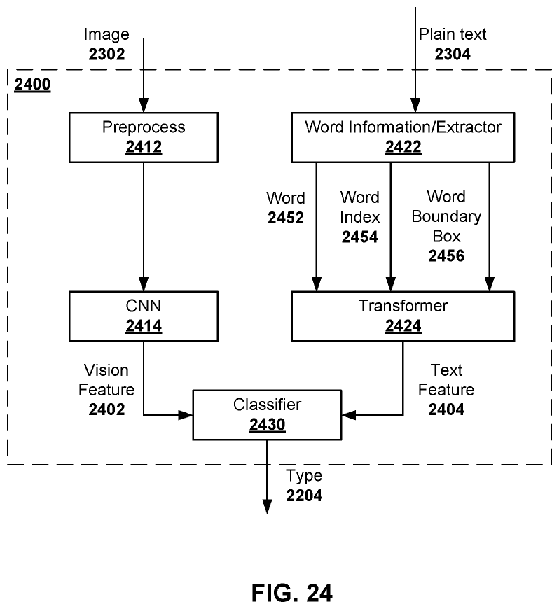

FIG. 24 illustrates an example of classification 2400, in accordance with some embodiments. In this example, word information extractor 2422 extracts words 2452, word indices 2454 and word boundary boxes 2456 from the plain text 2304 generated from OCR 2324. The transformer (or model) 2424 takes all this information and generates the text feature 2404 for the entire document. For example, the transformer (or model) 2424 may include an encoding process to encode text into a value that matches a document type. At the same time, image 2302 is processed 2412 according to the configuration of convolutional neural network (CNN) 2414, which generates the vision feature 2402. With the input of text feature 2404 and vision feature 2402, the classifier 2220 will predict the type for the document/image 2204.

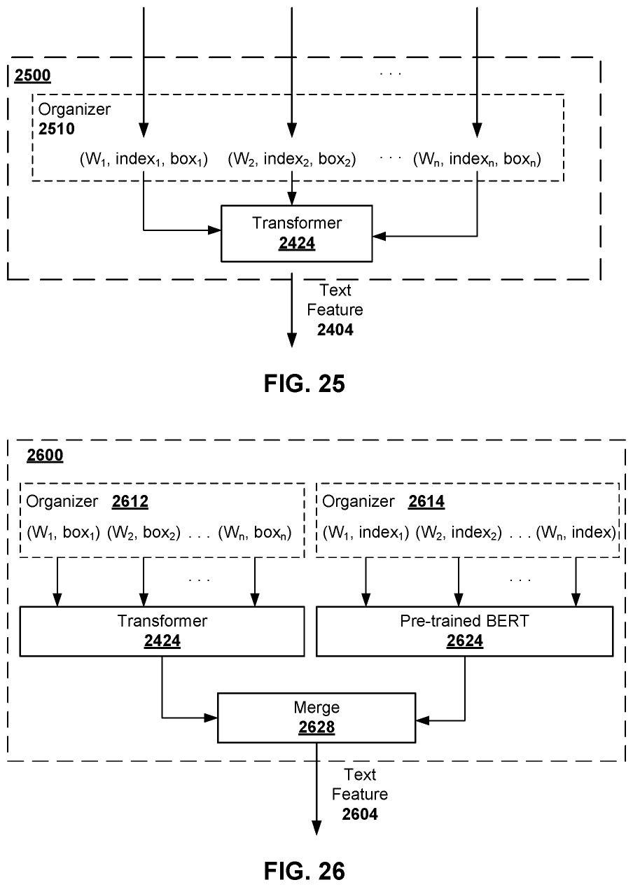

FIG. 25 illustrates an example of a transformer 2500, in accordance with some embodiments. Transformer 2500 includes a logical organizer 2510 and the transformer 2424. When getting the word list 2452, word index list 2454 and word boundary box list 2456, word organizer 2510 reorganizes the three lists such that the first input takes the first word from 2452, the first word index from 2454 and the first word box from 2456, and the second input takes the second word from 2452, the second word index from 2454 and the second word box from 2456, and so on. Suppose there are n words, then there will be n inputs to the transformer 2424. In some embodiments, transformer 2424 may be a kind of self-attention neural network. It should be understood that logical organizer 2510 may be implemented in extractor 2422, in transformer 2424 or between the extractor 2422 and transformer 2424.

FIG. 26 illustrates another example of a transformer 2600, in accordance with some embodiments. Transformer 2600 includes logical organizers 2612 and 2614, the transformer 2424, a pre-trained BERT 2624, and a merge function 2628. Organizer 2614 takes the first word from the word list 2452 and the first word index from the word index list 2454 as the first input to a pre-trained BERT language model 2624, and takes the second word from the word list 2452 and the second word index from the word index list 2454 as the second input to BERT model 2624, and so on. The pre-trained BERT 2624 will generate a feature vector. Organizer 2612 takes the first word from the word list 2452 and the first word box from the word box list 2456 as the first input to transformer 2424, and takes the second word from the word list 2452 and the second word box from the word box list 2456 as the second input to transformer 2424, and so on. The transformer 2424 will generate another feature vector. The two feature vectors will be merged 2628 and output the text feature 2604. It should be understood that logical organizer 2612 may be implemented in extractor 2422, in transformer 2424 or between the extractor 2422 and transformer 2424. It should be understood that logical organizer 2614 may be implemented in extractor 2422, in pre-trained BERT 2624 or between the extractor 2422 and pre-trained BERT 2624.

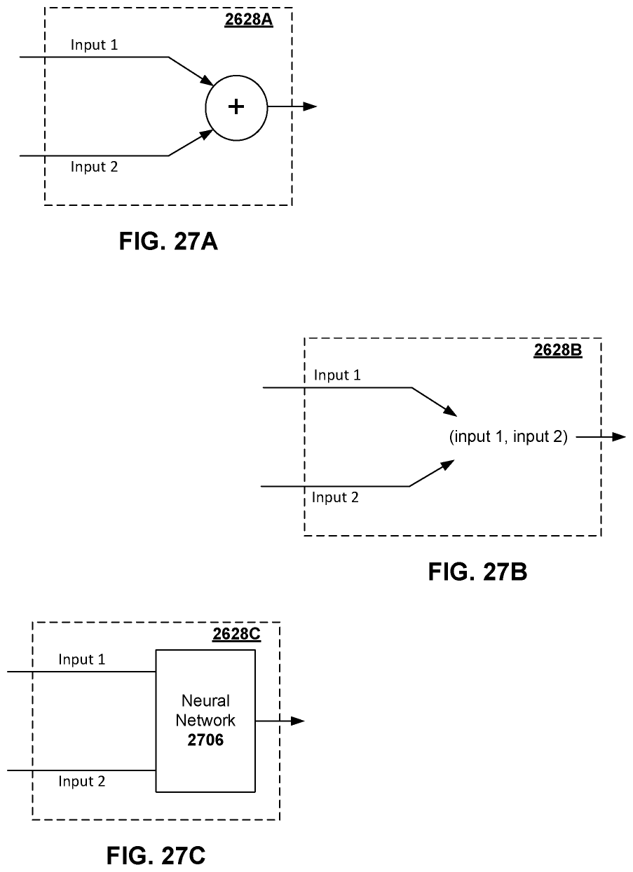

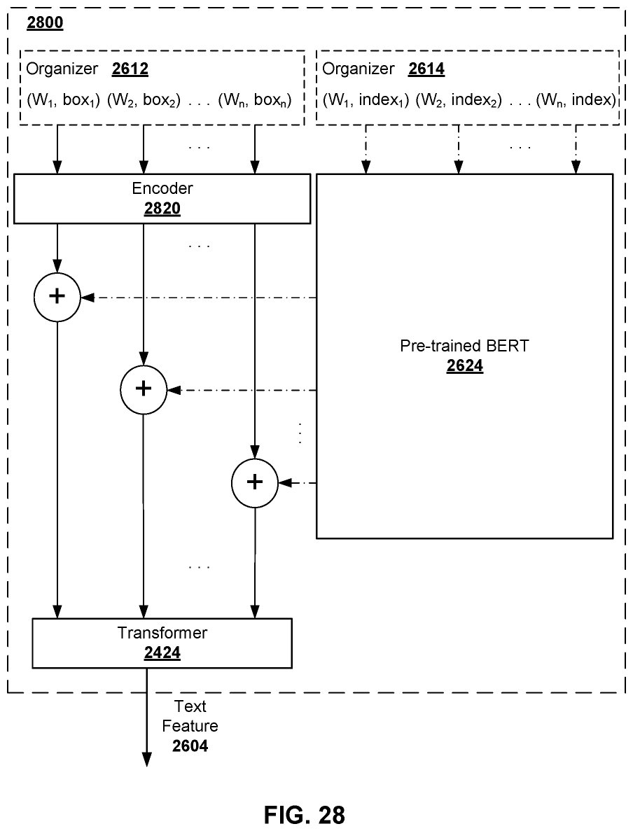

The merge operation 2628 could be anyone among FIGS. 27A to 27C, which illustrate examples of a merge operation 2628A to 2628C, in accordance with some embodiments. In merge operation 2628A, the two input vectors are added element-wise. In merge operation 2628B, the two input vectors are concatenated. In merge operation 2628C, the two vectors are input into another neural network 2706, which could be either a fully-connected one layer network or a deeper neural network.

FIG. 28 illustrates another example of a transformer 2800, in accordance with some embodiments. Transformer 2800 includes logical organizers 2612 and 2614, an ecoder 2820, the transformer 2424, and the pre-trained BERT 2624. The pre-trained BERT model 2624 takes the input from organizer 2614 and generates a list of feature vectors. These feature vectors will be added vector-wise with another list of vectors that are encoded from the input from organizer 2612 by the encoder 2820. The sum output are input into the transformer 2424 that generates the text feature 2604. In some embodiments, encoder 2850 could be another neural network. It should be understood that logical organizer 2612 may be implemented in extractor 2422, in encoder 2820 or between the extractor 2422 and encoder 2820.

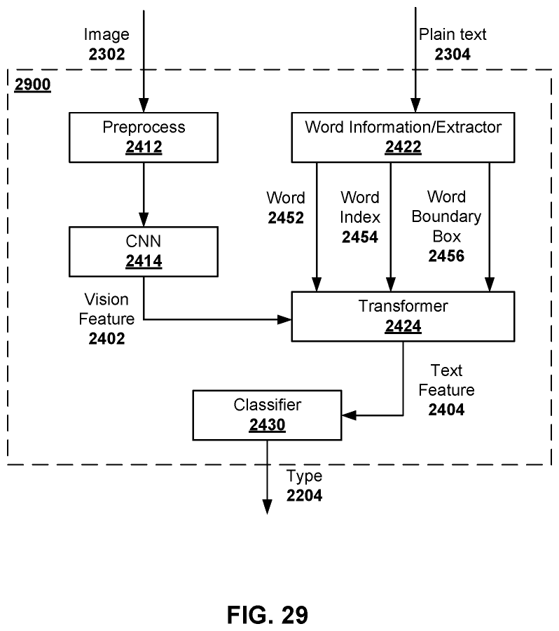

FIG. 29 illustrates another example of a classification unit 2900, in accordance with some embodiments. The processed image 2412 of the image 2302 is input into a CNN 2414 to generate its vision feature 2402, which together with the list of words 2452, the list of word indices 2454 and the list of word boundary boxes 2456, is ingested by the transformer 2424. The output of the transformer 2424 is input to the classifier 2430 to predict the type 2204 of the document/image.

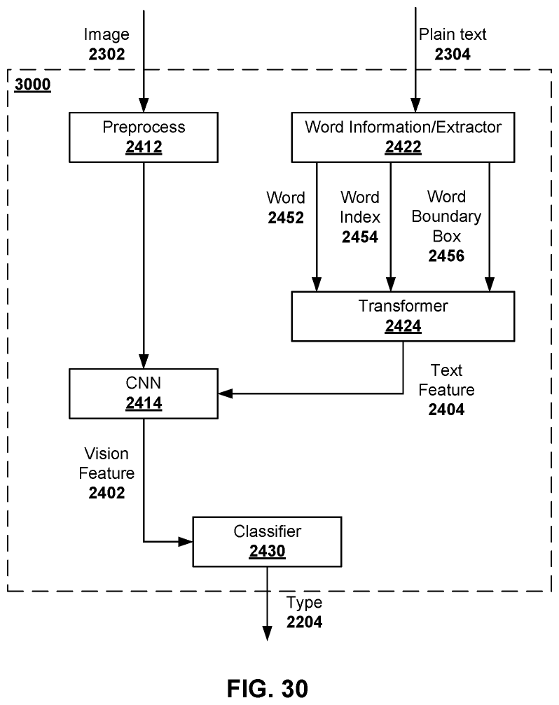

FIG. 30 illustrates another example of classification unit 3000, in accordance with some embodiments. The list of words 2452, the list of word indices 2454 and the list of word boundary boxes 2456 are input to the transformer 2424, and its output and the processed image 2412 are feed into a CNN 2414. The output of the CNN 2414 is used by the classifier 2430 to predict the type 2204 of the document/image.

The examples of FIGS. 28, 29 and 30 are in the word-level in text and the entire image level. More specifically, 2452, 2454 and 2456 are the lists of separate words, word index and word boundary box; the image is the whole page of the document/image.

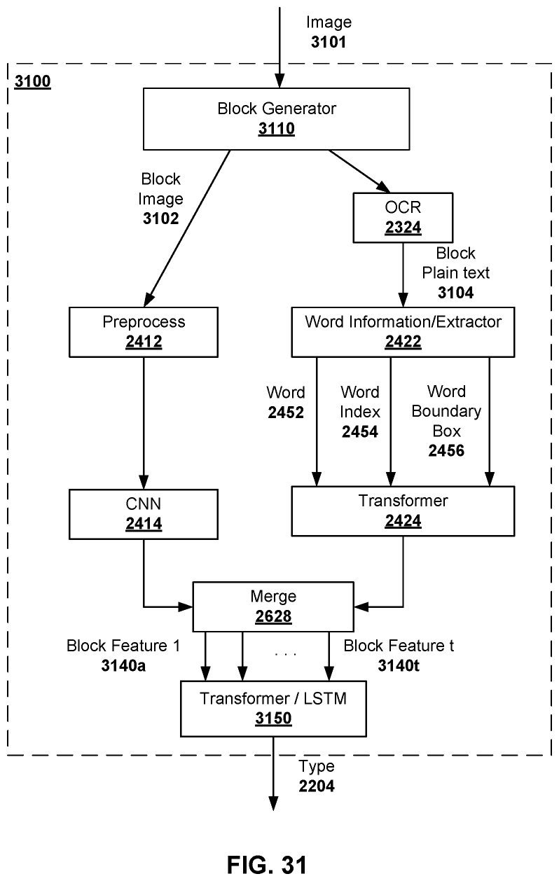

FIG. 31 illustrates, in a block-level view, another example of classification unit 3100, in accordance with some embodiments. The image 3101 is feed into the block generator 3110 and outputs a list of blocks and their boundary boxes. The block generator 3110 could be an object detector or something predefined based on rules. The block could be a sub-region of a logo, signature, handwriting, printed text, figure, and so on. From the block's boundary box, a sub-region 3102 of the image 3101 is cropped. Applying the same methodologies described above with respect to FIG. 28, FIG. 29 and FIG. 30 to the sub-region 3101 of each block from the block list, there are the same length of the list of block features 3140a to 3140t generated, block feature 1, block feature 2, . . . , block feature t, if there are t blocks generated from 3102. The list of block features 3102 along with their corresponding boundary boxes and/or block indices is feed into the transformer or long short-term memory network (LSTM) 3150 to predict the type 2204 of the document/image 3101. It should be understood that transformer 2424 is used to determine text within a single page, whereas transformer/LSTM 3150 is used to determine text among a group of pages. The transformer/LSTM 3150 may determine the relationship of the group of pages to classify the group of pages together.

### Intelligent Optical Character Recognition Platform

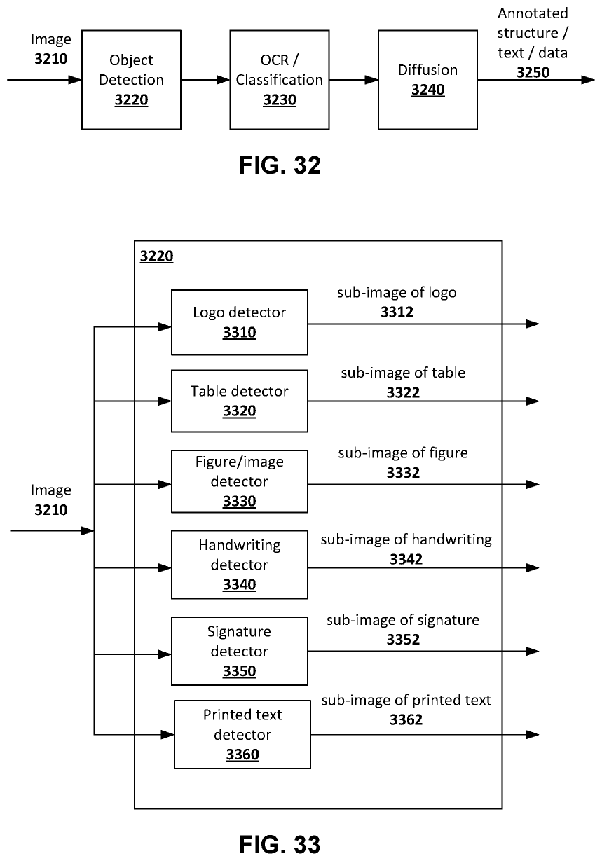

FIG. 32 illustrates, in a high-level diagram, an example of an optical character recognition platform 3200, in accordance with some embodiments. For a given image 3210, the object detection 3220 detects multiple objects from the input image, and the detected objects are processed separately by OCR and/or classification 3230. The output of the OCR/Classification unit 3232 could be plain text, data, image, etc., and the output is diffused by diffusion unit 3240 to generate an annotated structured text/data 3250. In some embodiments, a form comprises different sections where each section includes different features. Each section may be divided into block where each block is processed separately and then merged together by the diffusion unit 3240 to generate the annotated structured text/data 3250.

FIG. 33 illustrates an example of an object detection unit 3220, in accordance with some embodiments. In this example, the unit 3220 is module-based, and a module can be added or removed from the unit 3220. FIG. 33 shows an example of a list of modules, including a logo detector 3310, a table detector 3320, a figure/image detector 3330, a handwriting detector 3340, a signature detector 3350 and a printed text detector 3360. The list of modules generates a corresponding list of sub-images including a sub-image of a logo 3312, a sub-image of a table 3322, a sub-image of a FIG. 3332, a sub-image of a handwriting sample 3342, a sub-image of a signature 3352 and a sub-image of printed text 3362, respectively. It should be understood that each module may use built-in data set patterns or machine learning to detect and output their corresponding sub-image.

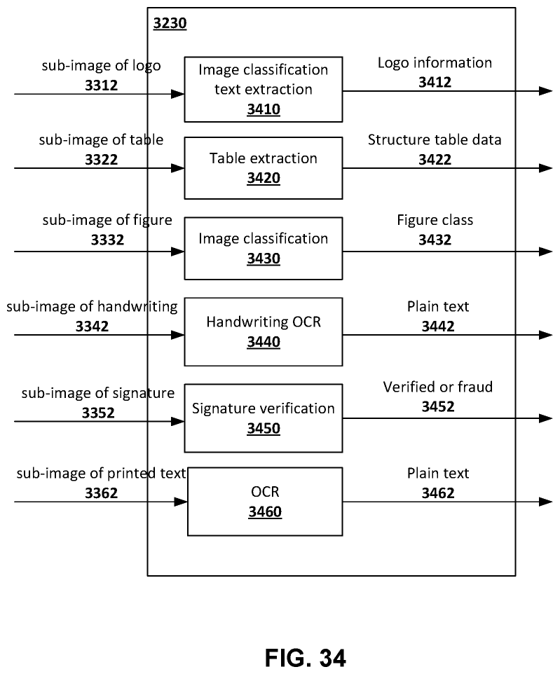

FIG. 34 illustrates an example of an OCR and/or classification unit 3230, in accordance with some embodiments. This example is also module-based, and a module can be added or removed from the unit 3230. A sub-image from the output of OCR/Classification unit 3230 may be processed by one or more modules. The sub-image of a logo 3312 can be input into different image classification or different text extraction 3410, such as OCRs, to obtain logo information 3412. The sub-image of a table 3322 may be processed by a table extractor 3420 to generate structured table data 3422. Figures 3332 may be classified 3430 into predefined classes 3432, such as flow chart, histogram, pie chart and so on. The sub-image of handwriting 3342 may be OCRed by a handwriting OCR module 3440 that outputs plain text 3442. The sub-image of a signature 3352 may be verified by a signature verifier 3450 to determine if the signature is valid or a fraud/fake 3452. The sub-image of printed text 3362 may be OCRed to output plain text 3462.

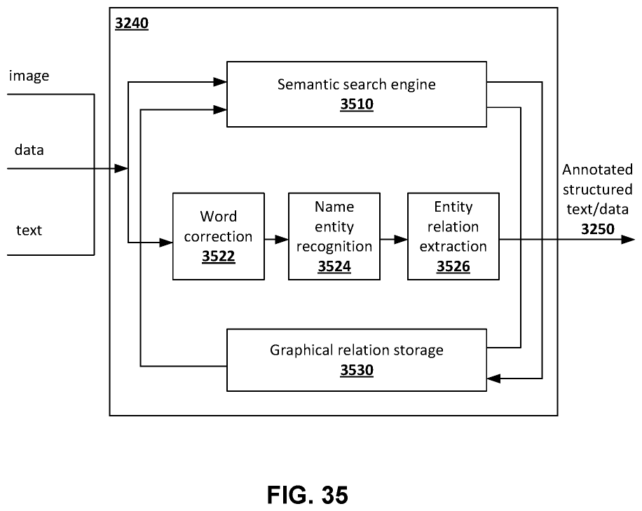

FIG. 35 illustrates an example of a diffusion unit 3240, in accordance with some embodiments. The input to the diffusion unit 3240 includes images, data and text. Text will be cleaned and corrected by looking up a dictionary or via NLP tools 3522. The cleaned text will be ingested by the name entity recognition 3524 to generate a list of entities, such as person, date, organization and so on. The cleaned text and the list of entities will be feed into the relation extraction module 3526 to output the relations between entities. For example, the model may extract and output a key value pair for each entity and its relation. All extracted entities and relations can be saved into a graphical relation storage 3530, and this storage 3530 may be the semantic engine for the semantic search engine 3510. Images and texts can be used as queries to the semantic search engine 3510. In some embodiments, the search engine 3512 can access public dataset on the web or proprietary dataset. Structured text and/or data 3250 is generated for the input image, including related annotations, search results and http links. For example, the structured text/data 3250 for a form may comprise an ordered listing of key value pairs associated with each entity on the form.

The discussion provides example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the foregoing discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements.

Although the embodiments have been described in detail, it should be understood that various changes, substitutions and alterations can be made herein.

Moreover, the scope of the present application is not intended to be limited to the particular embodiments of the process, machine, manufacture, composition of matter, means, methods and steps described in the specification.

As can be understood, the examples described above and illustrated are intended to be exemplary only.

1. A document index generating system comprising:
at least one processor; and
a memory storing a sequence of instructions which when executed by the at least one processor configure the at least one processor to:
preprocess a plurality of pages into a collection of data structures, each data structure comprising a representation of data for a page of the plurality of pages, the representation comprising at least one region on the page, the at least one processor configured to:
for each page, normalize the plurality of pages into a collection of images and a collection of plain text;
for each page, obtain vision features from the collection of images; and
for each page, process the collection of plain text;
classify each preprocessed page into at least one document type;
segment groups of classified pages into documents; and
generate a page and document index for the plurality of pages based on the classified pages and documents.

2. The document index generating system as claimed in claim 1, wherein to preprocess the collection of plain text, the at least one processor is configured to at least one of:
(i) perform optical character recognition (OCR) to the collection of plain text;
(ii) extract a word list, a word index list and a word boundary box list from the collection of plain text; or
(iii) generate arrays of entries comprising, for each indexed item in the word list, word index list and word boundary box list, an indexed word, an indexed word index and an indexed word boundary box; and
pass the arrays of entries to a transformer to extract text features.

3. The document index generating system as claimed in claim 1, wherein to obtain vision features of the collection of images, the at least one processor is configured to pass images of the plurality of pages through a convolution neural network.

4. (canceled)

5. (canceled)

6. The document index generating system as claimed in claim 1, wherein to preprocess the collection of plain text, the at least one processor is configured to extract a word list, a word index list and a word boundary box list from the collection of plain text, wherein the at least one processor is configured to:
generate a first set of arrays of entries comprising, for each indexed item in the word list and word boundary box list, an indexed word and an indexed word boundary box;
generate a second set of arrays of entries comprising, for each indexed item in the word list and word index list, an indexed word and an indexed word index;
pass the first set of arrays to a transformer;
pass the second set of arrays to a pre-trained BERT model; and
merge the results from the transformer and the pre-trained BERT model.

7. The document index generating system as claimed in claim 1, wherein preprocessing the plurality of pages into a collection of data structures comprises:
for each page in the plurality of pages:
converting that page to a bit map file format;
determining regions on that page based on at least one of:
the location of the region on the page;
the content in the region; or
the location of the region in relation to other regions on the page;
converting each region of that page into a machine-encoded content;
collecting the regions and corresponding content for that page into a data structure for that page; and
merging the page data structures into the collection of data structures.

8. The document index generating system as claimed in claim 7, wherein determining regions on that page comprises:
searching sections of the page for text or other items, the section comprising at least one of:
a top third of the page;
a middle third of the page;
a bottom third of the page;
a top quadrant of the page;
a bottom 15 percent of the page;
a bottom right corner of the page;
a top right corner of the page; or
the full page.

9. The document index generating system as claimed in claim 1, wherein classifying each preprocessed page into at least one document type comprises:
determining candidate document types for the page for each page in the collection of data structures.

10. The document index generating system as claimed in claim 9, wherein determining the candidate document type for the page comprises:
determining confidence score values for each candidate document type based on at least one of:
a presence of a combination of regions on the page; or
content in at least one of:
a region category types for each region on the page;
a title of the page;
an origin of the page;
a date of the page; or
a summary of the page.

11. The document index generating system as claimed in claim 1, wherein segmenting groups of pages into documents comprises:
clustering contiguous pages based on at least one of:
similar document types;
similar document titles; or
sequential page numbers.

12. The document index generating system as claimed in claim 1, comprising:
analyzing characteristics of the pages and documents to update missing information in the page and document index

13. A computer-implemented method of generating an index of a document, the method comprising:
preprocessing a plurality of pages into a collection of data structures, each data structure comprising a representation of data for a page of the plurality of pages, the representation comprising at least one region on the page, comprising:
for each page, normalizing the plurality of pages into a collection of images and a collection of plain text;
for each page, obtaining vision features from the collection of images; and
for each page, processing the collection of plain text;
classifying each preprocessed page into at least one document type;
segmenting groups of classified pages into documents; and
generating a page and document index for the plurality of pages based on the classified pages and documents.

14. The method as claimed in claim 13, wherein preprocessing the collection of plain text comprises at least one of:
(i) performing optical character recognition (OCR) to the collection of plain text;
(ii) extracting a word list, a word index list and a word boundary box list from the collection of plain text; or
(iii) generating arrays of entries comprising, for each indexed item in the word list, word index list and word boundary box list, an indexed word, an indexed word index and an indexed word boundary box; and
passing the arrays of entries to a transformer to extract text features.

15. The method as claimed in claim 13, wherein obtaining vision features of the collection of images comprises passing images of the plurality of pages through a convolution neural network.

16. (canceled)

17. (canceled)

18. The method as claimed in claim 13, wherein preprocessing the collection of plain text comprises extracting a word list, a word index list and a word boundary box list from the collection of plain text, comprising:
generating a first set of arrays of entries comprising, for each indexed item in the word list and word boundary box list, an indexed word and an indexed word boundary box;
generating a second set of arrays of entries comprising, for each indexed item in the word list and word index list, an indexed word and an indexed word index;
passing the first set of arrays to a transformer;
passing the second set of arrays to a pre-trained BERT model; and
merging the results from the transformer and the pre-trained BERT model.

19. The method as claimed in claim 13, wherein preprocessing the plurality of pages into a collection of data structures comprises:
for each page in the plurality of pages:
converting that page to a bit map file format;
determining regions on that page based on at least one of:
the location of the region on the page;
the content in the region; or
the location of the region in relation to other regions on the page;
converting each region of that page into a machine-encoded content;
collecting the regions and corresponding content for that page into a data structure for that page; and
merging the page data structures into the collection of data structures.

20. The method as claimed in claim 19, wherein determining regions on that page comprises:
searching sections of the page for text or other items, the section comprising at least one of:
a top third of the page;
a middle third of the page;
a bottom third of the page;
a top quadrant of the page;
a bottom 15 percent of the page;
a bottom right corner of the page;
a top right corner of the page; or
the full page.

21. The method as claimed in claim 13, wherein classifying each preprocessed page into at least one document type comprises:
determining candidate document types for the page for each page in the collection of data structures.

22. The method as claimed in claim 21, wherein determining the candidate document type for the page comprises:
determining confidence score values for each candidate document type based on at least one of:
a presence of a combination of regions on the page; or
content in at least one of:
a region category types for each region on the page;
a title of the page;
an origin of the page;
a date of the page; or
a summary of the page.

23. The method as claimed in claim 13, wherein segmenting groups of pages into documents comprises:
clustering contiguous pages based on at least one of:
similar document types;
similar document titles; or
sequential page numbers.

24. The method as claimed in claim 13, comprising:
analyzing characteristics of the pages and documents to update missing information in the page and document index.
